(19) United States
(12) Patent Application Publication
(10) Pub. No.: US 2025/0248814 A1
(43) Pub. Date: Aug. 7, 2025

(54) BALLOON ASSEMBLIES WITH INFLATABLE TOROID BALLOONS

(71) Applicant: EDWARDS LIFESCIENCES CORPORATION, Irvine, CA (US)

(72) Inventors: Ilan Leshecz, Hadera (IL); Anatoly Dvorsky, Haifa (IL)

(73) Assignee: EDWARDS LIFESCIENCES CORPORATION, Irvine, CA (US)

(21) Appl. No.: 19/033,058

(22) Filed: Jan. 21, 2025

Related U.S. Application Data
(63) Continuation of application No. PCT/US2023/028604, filed on Jul. 25, 2023.
(60) Provisional application No. 63/392,828, filed on Jul. 27, 2022.

(51) Int. Cl. A61F 2/24; A61M 25/10; A61M 39/24

(52) U.S. Cl. CPC A61F 2/2433; A61M 25/1002; A61M 25/1011; A61M 39/24; A61M 2025/1061; A61M 2025/1097; A61M 2039/2446; A61M 2205/0216; A61M 2205/3331; A61M 2210/12

(57) ABSTRACT

The present disclosure relates to balloon assemblies designed to permit blood flow through a central perfusion channel, at least when blood flows in a proximally-oriented direction. Such a balloon assembly can include one or more toroid balloons, each defining a cavity that can be independently fed by one or more inflation tubes coupled thereto to inflate the balloon. When the toroid balloons are inflated within a patient lumen or a prosthetic device, such as a prosthetic heart valve, blood is allowed to flow through the perfusion lumen. A balloon assembly can further include a flexible valve configured to regulate blood flow when the balloon assembly is inflated, to allow proximally directed flow through a channel formed in a flexible funnel thereof, and prevent backflow in the opposite direction.

### CROSS-REFERENCE TO RELATED APPLICATIONS

This application is a continuation of International Application No. PCT/US2023/028604, filed Jul. 25, 2023, which claims the benefit of U.S. Provisional Application No. 63/392,828, filed Jul. 27, 2022, which is incorporated by reference herein.

### FIELD

The present disclosure relates to apparatuses and methods that can be used in the treatment of heart valve disease, including balloon valvuloplasty and the delivery of transcatheter heart valves.

### BACKGROUND

Heart valve disease is a serious problem that involves the malfunction of one or more valves of the heart. The malfunction can manifest itself in a variety of manners. For example, valve stenosis is the calcification or narrowing of a native heart valve. As a result, the native heart valve is not able to completely open and blood flow through the native valve is impeded or restricted. Another example of heart valve disease is valve insufficiency. Valve insufficiency is the failure of a native heart valve to close properly to prevent leaking, or backflow, of blood through the valve.

Various methods have been developed to treat heart valve disease. Some of these methods require a balloon member that is expanded within the native heart valve. For example, a balloon member can be used in a valvuloplasty procedure where the balloon member is positioned within the native heart valve and expanded to increase the opening size (i.e., flow area) of the native heart valve and thereby improve blood flow. Another procedure that can be performed is a valve replacement, in which a native heart valve is replaced by an artificial heart valve. The implantation of an artificial heart valve in the heart can also involve the expansion of a balloon member in the valve annulus. For example, the balloon member can be used to increase the size of the native valve prior to implantation of the artificial valve and/or it can be used to expand and deploy the artificial valve itself.

### SUMMARY

The expansion of a balloon member within a native valve or other vascular passageway can temporarily block or restrict blood flow through the passageway. If blood flow is blocked or restricted in the passageway for too long, serious injury or death can occur. Furthermore, in the case of valve replacement, the positioning of the artificial heart valve may be complicated by the buildup of pressure in the left ventricle. Accordingly, valvuloplasty and valve replacement procedures, and other similar procedures which utilize expandable balloon members, must generally be performed quickly and/or with a heart pacing procedure, so that the balloon member is inflated for only a brief period. The present disclosure is directed toward inflatable balloon assemblies comprising series of toroid balloons that permit perfusion of blood through a perfusion lumen when the balloons are inflated in a passageway such as a patient lumen or a prosthetic valve.

According to one aspect of the disclosure, a delivery apparatus comprises a balloon assembly comprising a plurality of toroid balloons collectively defining a perfusion lumen along a longitudinal axis of the balloon assembly.

In some aspects, each toroid balloon in configured to transition between a deflated state and an inflated state.

In some aspects, each toroid balloon comprises a balloon wall enclosing a cavity, and at least one balloon opening extending through a thickness of the balloon wall.

In some aspects, the delivery apparatus further comprises at least one inflation tube.

In some aspects, the at least one inflation tube comprises at least one tube opening which is in fluid communication with the cavity of at least one of the plurality of toroid balloons.

In some aspects, when the toroid balloons are in the inflated state, the perfusion lumen is configured to allow flow therethrough.

In some aspects, at least one of the at least one inflation tube is in fluid communication with the cavities of more than one of the plurality of toroid balloons.

In some aspects, the at least one inflation tube comprises a plurality of inflation tubes.

In some aspects, at least one of the at least one inflation tube extends through the perfusion lumen.

In some aspects, the perfusion lumen defines a perfusion lumen diameter which is greater in the inflated state than in the deflated state.

In some aspects, each toroid balloon defines a toroidal outer diameter which is greater in the inflated state than in the deflated state.

In some aspects, at least two of the toroid balloons define different toroidal outer diameters in the inflated state.

In some aspects, at least one of the at least one inflation tube extends through at least one of the plurality of toroid balloons.

In some aspects, the delivery apparatus further comprises a flexible valve disposed within the perfusion lumen.

In some aspects, the flexible valve comprises a valve base and a flexible funnel defining a channel extending proximally from a funnel inlet at the valve base to a funnel outlet and defining a funnel axis therealong.

In some aspects, the valve base is attached at an outer circumference thereof to at least one of the plurality of toroid balloons.

In some aspects, the flexible valve is configured to transition between an open state in which proximally oriented flow can pass through the channel, and a closed state in response to distally oriented backflow such that the flow direction changes the state of the flexible valve.

According to one aspect of the disclosure, a delivery apparatus comprises a balloon assembly. The balloon assembly comprises a toroid balloon that defines a perfusion lumen around a longitudinal axis of the balloon assembly, and a flexible valve disposed within the perfusion lumen.

In some aspects, the toroid balloon in configured to transition between a deflated state and an inflated state.

In some aspects, the toroid balloon comprises a balloon wall enclosing a cavity, and at least one balloon opening extending through a thickness of the balloon wall.

In some aspects, the flexible valve comprises a valve base and a flexible funnel.

In some aspects, the valve base is attached at an outer circumference thereof to the toroid balloon.

In some aspects, the flexible funnel defines a channel extending proximally from a funnel inlet at the valve base to a funnel outlet and defining a funnel axis therealong.

In some aspects, the delivery apparatus further comprises at least one inflation tube comprising at least one tube opening exposed to and in fluid communication with the cavity.

In some aspects, the flexible valve is configured to transition between an open state in which proximally oriented flow can pass through the channel, and a closed state in response to distally oriented backflow such that the flow direction changes the state of the flexible valve

In some aspects, the valve base comprises a flexible membrane.

In some aspects, the at least one inflation tube comprises a plurality of inflation tubes.

In some aspects, the at least one inflation tube extends through the perfusion lumen.

In some aspects, at least one of the at least one inflation tube extends through the toroid balloon.

According to one aspect of the disclosure, a method for inflating a balloon assembly inside a treatment site comprises delivering the balloon assembly, which comprises a plurality of toroid balloons in a deflated state thereof, through the vasculature of a patient to the treatment site, and inflating the toroid balloons within a patient lumen.

In some aspects, each of the toroid balloons comprises a cavity in fluid communication with at least one inflation tube.

In some aspects, inflating the toroid balloon comprises forming a perfusion lumen configured to permit blood flow therethrough.

In some aspects, the perfusion lumen allows blood flow at least in a proximally oriented direction.

In some aspects, inflating the toroid balloons to permit blood to flow through the perfusion lumen is performed without rapid pacing.

In some aspects, each toroid balloon defines a toroidal outer diameter, and wherein inflating the toroid balloons comprises increasing the toroidal outer diameters such that outer sides of balloon walls of at least some of the toroid balloons press against an inner surface of the patient lumen.

In some aspects, each toroid balloon defines a toroidal outer diameter, and wherein inflating the toroid balloons comprises increasing the toroidal outer diameters such that outer sides of balloon walls of at least some of the toroid balloons press against an inner surface of a prosthetic device disposed around the balloon, thereby expanding the prosthetic device against an inner wall of the patient lumen.

In some aspects, the balloon assembly further comprises a flexible valve disposed within the perfusion lumen.

In some aspects, the flexible valve comprises a valve base attached at an outer circumference thereof to at least one of the plurality of toroid balloons, and a flexible funnel defining a channel extending proximally from a funnel inlet at the valve base to a funnel outlet and defining a funnel axis therealong.

In some aspects, the method further comprises aligning the funnel axis parallel to a longitudinal axis of the balloon assembly when blood flows in a proximally oriented direction, thereby permitting blood to pass through the channel.

In some aspects, the method further comprises folding the flexible funnel in response to backflow in the distal direction, such that the funnel axis is angled relative to the longitudinal axis, thereby preventing blood from flowing back in the distal direction through the channel.

According to some aspects of the disclosure, there is provided a delivery apparatus comprising a balloon assembly comprising at least one toroid balloon and at least one inflation tube. The at least one toroid balloon defines a perfusion lumen along a longitudinal axis of the balloon assembly. The at least one toroid balloon in configured to transition between a deflated state and an inflated state, and comprises a balloon wall enclosing a cavity, and at least one balloon opening extending through a thickness of the balloon wall. The at least one inflation tube comprises at least one tube opening which is in fluid communication with the cavity of the at least one toroid balloon. When the at least one toroid balloon is in the inflated state, the perfusion lumen is configured to allow flow therethrough.

In some examples, the perfusion lumen is configured to allow flow therethrough in a proximally oriented direction.

In some examples, the perfusion lumen is configured to allow blood flow therethrough when the at least one toroid balloon is in the inflated state within a patient lumen.

In some examples, the perfusion lumen is configured to allow blood flow therethrough when the at least one toroid balloon is in the inflated state within a prosthetic device.

In some examples, the perfusion lumen is configured to allow blood flow therethrough when the at least one toroid balloon is in the inflated state within a prosthetic heart valve.

In some examples, the perfusion lumen is configured to allow blood flow therethrough when the at least one toroid balloon is in the inflated state within a lumen of a testing apparatus.

In some examples, the at least one toroid balloon comprises a plurality of toroid balloons.

In some examples, the plurality of toroid balloons are attached to each other.

In some examples, the delivery apparatus further comprises a flexible valve disposed within the perfusion lumen. The flexible valve comprises a valve base and a flexible funnel. The valve base is attached, at an outer circumference thereof, to the at least one toroid balloon. The flexible funnel defines a channel extending proximally from a funnel inlet at the valve base to a funnel outlet. The flexible funnel defines a funnel axis therealong. The flexible valve is configured to transition between an open state in which proximally oriented flow can pass through the channel, and a closed state in response to distally oriented backflow such that the flow direction changes the state of the flexible valve

In some examples, the valve base is attached to at least one of a plurality of toroid balloons.

In some examples, the funnel axis is parallel to the longitudinal axis in the open state of the flexible valve.

In some examples, the flexible funnel is bent in the closed state of the flexible valve, such that the funnel axis is angled relative to the longitudinal axis.

According to some aspects of the disclosure, a method is provided for delivering and inflating the at least one toroid balloon of the delivery apparatus. The method comprises delivering the balloon assembly that comprises the at least one toroid balloon in a deflated state thereof, through the vasculature of a patient to a treatment site. The method further comprises inflating the at least one toroid balloon within a patient lumen, thereby forming the perfusion lumen that permits blood flow therethrough.

In some examples, blood is permitted to flow at least in a proximally oriented direction through a perfusion lumen.

In some examples, the balloon assembly further comprises a flexible valve disposed within the perfusion lumen. The flexible valve comprises a valve base and a flexible funnel. The valve base is attached to the at least one toroid balloon. The flexible funnel defines a funnel axis therealong

In some examples, the method further comprises aligning the funnel axis parallel to a longitudinal axis of the balloon assembly when blood flows in a proximally oriented direction, thereby permitting blood to pass through the channel of the flexible valve.

In some examples, the method further comprises folding the flexible funnel in response to backflow in the distal direction, such that the funnel axis is angled relative to the longitudinal axis, thereby preventing blood from flowing back in the distal direction through the channel.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 shows an exemplary delivery apparatus with a balloon assembly carrying a prosthetic device located along a distal end portion.

FIGS. 2A and 2B show sectional side views of an exemplary balloon assembly in deflated and inflated states of toroid balloons thereof.

FIG. 2C shows a view in perspective of a distal portion of a delivery apparatus equipped with the balloon assembly of FIG. 2B.

FIGS. 3A and 3B show a view in perspective and a sectional view, respectively, of a distal portion of a delivery apparatus with a plurality of inflation tubes coupled to toroid balloons of an exemplary balloon assembly.

FIGS. 4A and 4B show a view in perspective and a sectional view, respectively, of a distal portion of a delivery apparatus with an exemplary balloon assembly having proximal, central and distal sections.

FIG. 5 shows an exemplary balloon expandable prosthetic valve that can be used in combination with balloon assemblies of the current disclosure.

FIG. 6 shows a view in perspective or an exemplary arrangement in which each inflation tube is coupled to a different toroid balloon.

FIGS. 7A and 7B show a view in perspective and a sectional view, respectively, of an exemplary arrangement in which different inflation tubes are coupled to different pluralities of toroid balloons.

FIGS. 8A and 8B show a view in perspective and a sectional view, respectively, of an exemplary balloon assembly provided with a temporary flexible valve, illustrated in an open state.

FIGS. 9A and 9B show a view in perspective and a sectional view, respectively, of the balloon assembly of FIG. 8A-8B, with the temporary flexible valve illustrated in a closed state.

FIGS. 10A and 10B show a view in perspective and a sectional view, respectively, of a distal portion of a delivery apparatus with inflation tubes extending through cavities of toroid balloons of an exemplary balloon assembly, wherein at least one of the inflation tubes is in fluid communication with all toroid balloons.

FIGS. 11A and 11B show a view in perspective and a sectional view, respectively, of a distal portion of a delivery apparatus with inflation tubes extending through cavities of toroid balloons of an exemplary balloon assembly, wherein at least one of the inflation tubes is in fluid communication with some but not all toroid balloons.

FIGS. 12A and 12B show sectional side views of an exemplary balloon assembly of the type shown in FIGS. 11A-11B, further including a temporary flexible valve, illustrated in open and closed state, respectively.

FIGS. 13A and 13B illustrate stages of an exemplary method of the balloon utilized in a valvuloplasty procedure.

FIGS. 14A to 14C illustrate stages of an exemplary method of the balloon utilized in a prosthetic valve implantation.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/of” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

It should be understood that the disclosed examples can be adapted to deliver inflatable balloons, and in some examples, to deliver and implant prosthetic devices expandable by such inflatable balloons, to and/or in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

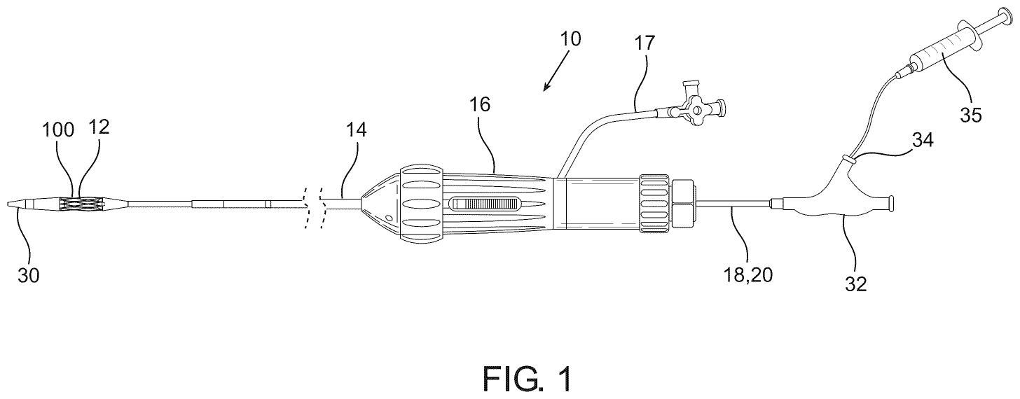

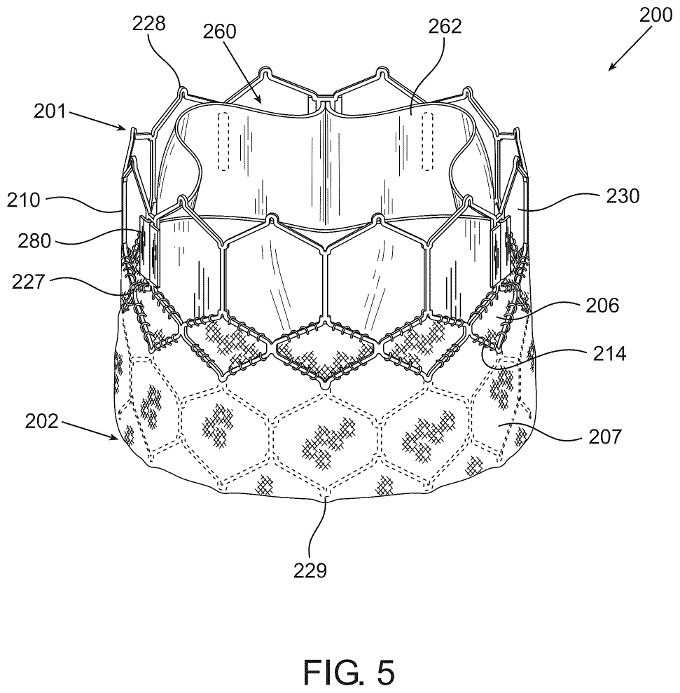

FIG. 1 illustrates a delivery apparatus 10, according to one configuration, adapted to deliver balloon assembly 100, optionally carrying a balloon expandable prosthetic device 12 thereon. Prosthetic device 12 can be a prosthetic valve, such as the prosthetic heart valve 200 described below and illustrated with respect to FIG. 5 or other types of prosthetic valves. It should be understood that the delivery apparatus 10 can be used to implant prosthetic devices other than prosthetic valves, such as stents or grafts, and that delivery apparatus 10 can be used to deliver an expandable balloon that does not necessarily carry an expandable prosthetic device, for example for medical procedures that can include valvuloplasty, pre-ballooning and post-ballooning.

The delivery apparatus 10 can generally include a steerable delivery shaft 14 and at least one inflation tube 20 extending through the delivery shaft 14. The delivery shaft 14 and the inflation tube(s) 20 can be adapted to slide longitudinally relative to each other to facilitate delivery and positioning of a prosthetic device 12 at an implantation site in a patient's body. When more than one inflation tube 20 is provided, the plurality of inflation tubes 20 are configured to move axially in unison, but may optionally deflect away from each other in the radial direction, at least along distal portions thereof. In some examples, delivery apparatus 10 further comprises a balloon assembly shaft 18, which can be optionally a multi-lumen shaft that defines a plurality of lumens through which a corresponding plurality of inflation tubes 20 extend. When a balloon assembly shaft 18 is provided, the delivery shaft 14 and the balloon assembly shaft 18 can be similarly adapted to slide longitudinally relative to each other to facilitate delivery and positioning of a prosthetic device 12 at an implantation site in a patient's body.

The delivery apparatus 10 includes a handle 16 and a balloon assembly 100 mounted on a distal end of the inflation tube(s) 20. The handle 16 can include a side arm 17 having an internal passage which fluidly communicates with a lumen defined by the handle 16. A balloon expandable prosthetic device 12, such as balloon expandable prosthetic heart valve 200, can be carried in a crimped state over the balloon assembly 100. The outer delivery shaft 14 can extend over the inflation tube(s) 20, and at least during delivery through a patient's vasculature, a distal portion of the delivery shaft 14 can extend over a deflated balloon assembly 100 as well. The delivery shaft 14 and the inflation tube(s) 20 can be adapted to slide longitudinally relative to each other to facilitate delivery and positioning of balloon assembly 100 and/or a prosthetic device 12 at a treatment site (or implantation site in case of a prosthetic device) in a patient's body.

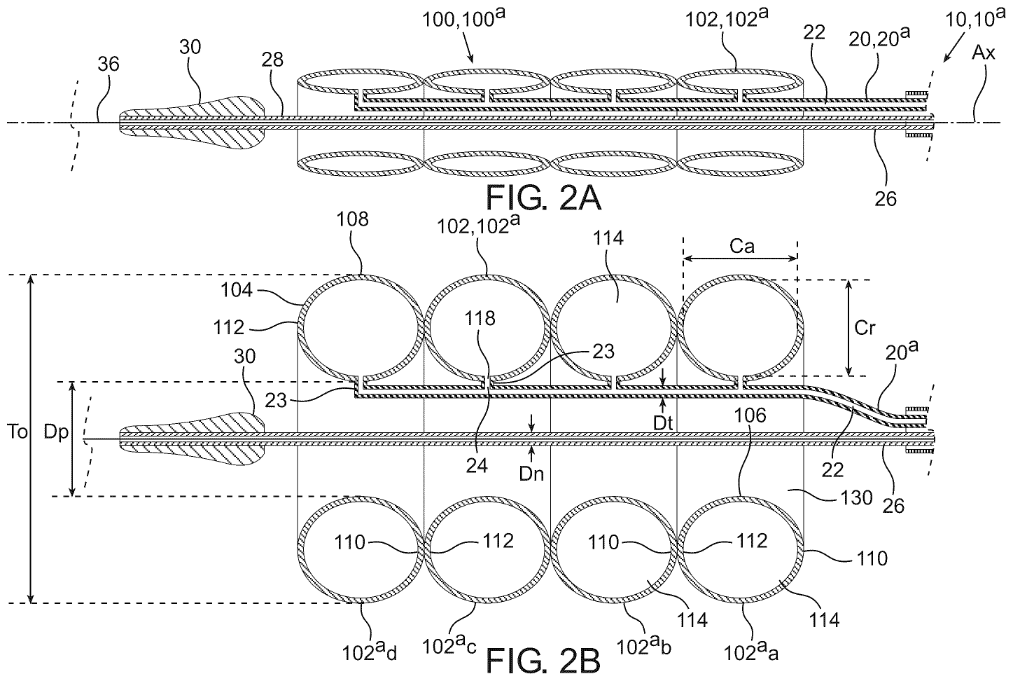

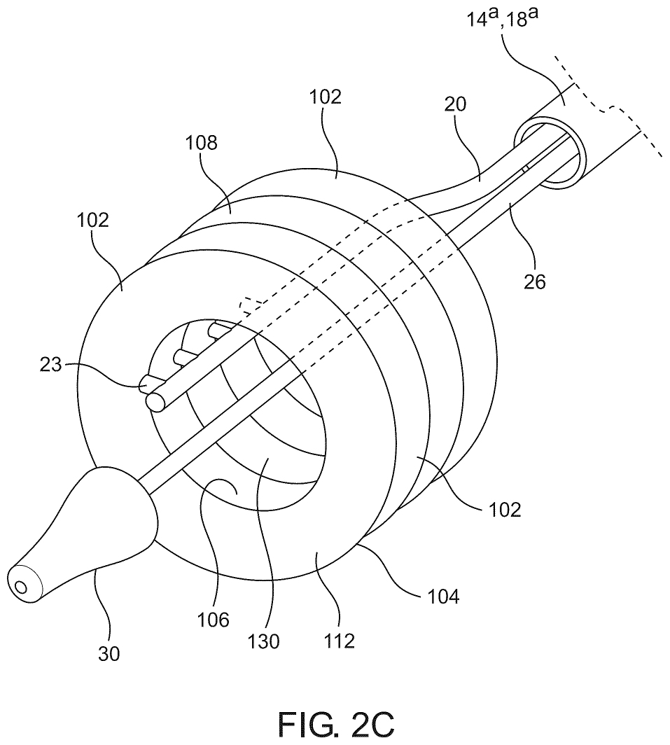

A nosecone 30 can be mounted at the distal end of the delivery apparatus 10 to facilitate advancement of the delivery apparatus 10 through the patient's vasculature to the site of treatment. The nosecone 30 can be connected to a separate elongated nosecone shaft 26 (shown in FIGS. 2A-2B, for example) so that nosecone 30 can move independently of other elements of delivery apparatus 10. Nosecone shaft 26 can extend through a lumen of the delivery shaft 14, substantially parallel to the at least one inflation tube 20. When a plurality of inflation tubes 20 are provided, the nosecone shaft 26 can extend between the inflation tubes 20. The inflation tube(s) 20 and the nosecone shaft 26 and the inflation tube(s) 20 can be adapted to independently move along the longitudinal direction relative to each other. When a balloon assembly shaft 18 is provided, the nosecone shaft 26 can extend therethrough, optionally next to the one or more inflation tube(s) 20. When the balloon assembly shaft 18 is provided as a multi-lumen shaft, it can further include a lumen through which the nosecone shaft 26 can pass. The nosecone shaft 26 and the inflation tube(s) 20 can be adapted slide longitudinally relative to each other.

The inflation tube(s) 20, and when provided, the balloon assembly shaft 18, can extend through the handle 16 and a proximal portion 32 which can be disposed proximally to the handle 16. The proximal portion 32 can be formed with a fluid passageway 34 that is fluidly connectable to a fluid source (e.g., saline) for inflating the balloon. The fluid source comprises an inflation fluid 35. The term “inflation fluid”, as used herein, means a fluid (e.g., saline) used for inflating balloon 100. Fluid passageway 34 is in fluid communication with inflation tube(s) 20, such that fluid from the fluid source can flow through fluid passageway 34, through tube lumen(s) 22 of the inflation tube(s) 20, and into balloons 102 comprised in balloon assembly 100 to inflate the balloons 102, and optionally deploy prosthetic device 12 if such a device is crimped over the balloon.

When more than one inflation tube is provided, the fluid passageway 34 can be split to form fluid communication with a plurality of inflation tubes 20, such as by including an internal manifold branched into a corresponding plurality of inflation tubes 20. Alternatively, fluid passageway 34 can include several independent passageways, each being in fluid communication with one or some of the inflation tubes 20. In such cases, fluid can be supplied independently to each passageway, such as by being connected to a separate fluid source (e.g., a separate syringe or a separate outlet of an inflation pump), allowing each inflation tube 20 or each subset of inflation tubes 20 to be independently utilized to deliver inflation tubes to balloons 102 or subset of balloons 102 mounted on the distal ends of the corresponding inflation tubes 20.

In FIG. 1, a prosthetic device 12 is mounted on the balloon assembly 100 and is shown in a crimped state, providing prosthetic device 12 with a reduced diameter for delivery to the heart via the patient's vasculature. As mentioned above, it should be understood that balloon assembly 100 can be configured for delivery to a treatment location without a prosthetic device (such as a prosthetic heart valve) mounted thereon, either for off-balloon delivery of the prosthetic device to a treatment location (as discussed below) or for use of the balloon in a valvuloplasty procedure.

Although the illustrated examples discussed herein refer to the prosthetic device (e.g., prosthetic valve) as being crimped or mounted on the balloon assembly for delivery to the treatment location, it should be understood that the prosthetic device can be crimped or mounted at a location different from the location of balloon assembly (e.g., proximal to the balloon assembly) and repositioned over the balloon assembly at some time before inflating the balloon assembly and deploying the prosthetic device. This off-balloon delivery allows the prosthetic device to be crimped to a lower profile than would be possible if the prosthetic device was crimped on top of the balloon assembly. The lower profile permits the physician to more easily navigate the delivery apparatus (including the crimped prosthetic device) through a patient's vasculature to the treatment location. The lower profile of the crimped prosthetic device can be particularly helpful when navigating through portions of the patient's vasculature which are particularly narrow, such as the iliac artery.

Balloon assembly 100 comprises a plurality of toroid balloons 102, wherein each toroid balloon 102 is configured to transition between a deflated state and an inflated state. FIG. 2A shows a sectional side view of a distal portion of delivery apparatus 10 with one exemplary balloon assembly 100^{a} that includes a series of balloons 102^{a}, shown in a deflated state. FIGS. 2B and 2C show a sectional side view and a view in perspective of the distal portion of apparatus 10 with the balloons 102^{a} of balloon assembly 100^{a} in the inflated state.

Each toroid balloon 102 comprises a balloon wall 104 defining an internal cavity 114. All of the toroid balloons 102 are circumferentially disposed around a longitudinal axis Ax of the balloon assembly 100, wherein each toroid balloon 102 assumes a toroid shape in its inflated state, as shown in FIG. 2C for example. The term “toroid”, as used herein with respect to individual balloons 102, relates generally to any annular, ring, or donut shaped body of the balloon 102 (including the shape of the balloon wall 104 and the corresponding cavity 114), regardless of cross-sectional geometry of the balloon 102.

Each toroid balloon 102, and more specifically, the balloon wall 104 of each toroid balloon 102 includes an inner side 106 facing the longitudinal axis Ax, an outer side 108 opposite to the inner side 106 and facing away from the longitudinal axis Ax (for example, toward an inner wall of a patient lumen 40 in which the balloon assembly 100 is inflated), and two lateral sides extending between the inner and the outer sides, wherein the lateral sides include a proximal side 110 oriented toward the proximal end of the delivery apparatus (such as toward handle 16), and an opposite distal side 112 facing toward the distal end of the delivery apparatus.

Conventional inflation procedures, particularly for replacement of an aortic valve, are usually performed under rapid pacing. Rapid pacing is a technique that involves electrical stimulation of the heart using pacemaker leads inserted into the heart. The heart rhythm of the patient is then accelerated to over 180 bpm which in fact causes the heart to flutter and thus not to effectually contract. While rapid pacing may help in toleration of the native valve's occlusion, the added procedure involves added risk, and a small number of patients do not tolerate accelerated pacing very well. In some rare instances, there can be long term myocardial damage due to extended rapid pacing. The balloon assembly 100 of the current disclosure defines a central perfusion lumen 130 that allows blood flow therethrough when the toroid balloons 102 are in the inflated state. This can allow the balloon assembly 100 to be utilized, in some examples, without rapid pacing. Thus, the balloon assembly 100 is designed to permit blood perfusion through the central perfusion lumen 130 in the inflated state of the toroid balloons 102. While exemplary balloon assembly 100^{a} is illustrated in FIGS. 2A-2C including a sequence of four toroid balloons 102^{a}, it is to be understood that this is shown by way of illustration and not limitation, and that a balloon assembly 100 can include any other number of toroid balloons 102.

Each toroid balloon 102 is in fluid communication with at least one inflation tube 20, such as via at least one tube opening 24 of the inflation tube 20, exposed to the cavity 114 of the corresponding balloon 102. The at least one inflation tube 20 can extend through perfusion lumen 130, in which case one or more tube openings 24 thereof can be formed as side openings, facing the respective inner side(s) 106 of toroid balloon(s) 102. In some examples, inflation tube 20 can include at least one side port 23 extending radially away from tube lumen 22 and terminating at tube opening 24. The cavity 114 of each toroid balloon 102 is completely sealed except for the tube opening(s) 24, such that there is no direct fluid communication between the cavities 114 of the different toroid balloons 102, except for each cavity 114 being in fluid communication with one or more tube lumen 22 of corresponding inflation tube(s) 20.

In the example illustrated in FIGS. 2A-2C, the exemplary delivery apparatus 10^{a} is shown to include a single inflation tube 20^{a} which is in fluid communication with all of the toroid balloons 102^{a}. The inflation tube 20^{a} extends through perfusion lumen 130, and is shown to include four side ports 23 extending between the tube lumen 22 and the corresponding tube openings 24 exposed to the cavities 114 of all four toroid balloons 102^{a}. Thus, inflation fluid supplied through the single inflation tube 20^{a} can fill all cavities 114 to inflate all of the toroid balloons 102^{a}.

Various exemplary implementations for delivery apparatus 10 and components thereof, such as balloon assemblies 100, inflation tubes 20, and the like, can be referred to, throughout the specification, with superscripts, for ease of explanation of features that refer to such exemplary implementations. It is to be understood, however, that any reference to structural or functional features of any apparatus, assembly or component, without a superscript, refer to these features being commonly shared by all specific exemplary implementations that can be also indicated by superscripts. In contrast, features emphasized with respect to an exemplary implementation of any apparatus, assembly or component, referred to with a superscript, may be optionally shared by some but not necessarily all other exemplary implementations.

In some examples, each toroid balloon 102 is coupled to at least one inflation tube 20. For example, the balloon wall 104 of each toroid balloon 102 can be attached to a corresponding side port 23, as illustrated in FIGS. 2A-2C. In some examples, the balloons 102 can be in direct contact with each other, without being spaced from each other along the longitudinal axis Ax. For example, at least one lateral side of each balloon can be in direct contact with at least one lateral side of an adjacent balloon. In some examples, the toroid balloons 102 can be attached to each other, such as being glued, welded, or otherwise bonded to each other at their lateral sides. In the example illustrated in FIGS. 2A-2C, the distal side 112 of balloon 102^{a}a is shown to be in contact with, and optionally attached to, the proximal side 110 of subsequent balloon 102^{a}b, and of the balloons 102^{a}b and 102^{a}c have both of their lateral sides in contact with, and optionally attached to, the lateral sides of the adjacent balloons 102 situated proximally and distally thereto, such that the proximal side 110 of the balloon 102^{a}d is similarly in contact with, and optionally attached to, the distal side 112 of balloon 102^{a}c.

As further shown in FIGS. 2A-2C, the nosecone shaft 26 extends through the perfusion lumen 130, and defines a guidewire lumen 28 extending along the entire length of the nosecone shaft 26 and nosecone 30, through which a guidewire 36 can pass, such that the entire delivery apparatus 10 can be advanced toward the treatment region over the guidewire 36. When inflation tube 20 also extends through perfusion lumen 130, the nosecone shaft 26 can be situated next to the inflation tube 20, or between inflation tubes 20 if a plurality of inflation tubes are provided. In the deflated state of balloons 102, as shown in FIG. 2A, the nosecone shaft 26 and inflation tube(s) 20 can be in close proximity to each other, and optionally even contacting each other, with the deflated balloons 102 disposed therearound, to result in a lower profile along balloon assembly 100. This lower profile permits the surgeon to more easily navigate the delivery apparatus (including deflated balloon assembly 100) through a patient's vasculature to the treatment location. The lower profile of the deflated balloon assembly is particularly helpful when navigating through portions of the patient's vasculature which are particularly narrow, such as the iliac artery.

Each toroid balloon 102 can include at least one balloon opening 116 extending through the thickness of balloon wall 104, aligned with a corresponding tube opening 24. In some examples, the at least one balloon opening 116 comprises a radially oriented opening 118 formed at the inner side of balloon wall 104, aligned with a corresponding tube opening 24, and optionally side port 23, of a tube lumen 22 extending through perfusion lumen 130.

As mentioned above, the tube lumen of any inflation tube 20 is in fluid communication with a fluid source (e.g., a syringe or a pump) that can inject an inflation fluid 35 (e.g., saline) into corresponding cavities 114 via tube opening 24. In this way, fluid from the fluid source can flow through the fluid passageway(s) 34, through tube lumen(s) 22, and into cavities 114 via tube openings 24 to inflate the toroid balloons 102 and optionally expand and deploy a prosthetic device 12 when such a device is disposed thereon. For example, the pressure of the fluid within toroid balloons 102 may provide the force that allows the balloon assembly 100 to dilate the prosthetic device 12 and/or surrounding anatomy. Further, the tube lumen(s) 22 may be configured to withdraw fluid from the cavities 114 through the tube opening 24 to deflate the toroid balloons 102.

With reference to FIG. 2B, when the toroid balloons 102 are inflated to assume their toroid shape, the diameter of the perfusion lumen 130, denoted Dp, is increased to allow blood flow therethrough. The perfusion lumen diameter Dp in the inflated state can be designed, in some examples, to be greater than the sum of diameters of any shafts and tubes that extend through perfusion lumen 130. For example, the nosecone shaft 26 can define an outer diameter Dn, and each inflation tube 20 can define an outer tube diameter Dt, such that the perfusion lumen diameter Dp is greater, in the inflated state, than the sum of the nosecone shaft diameter Dn and the tube diameter(s) Dt of inflation tube(s) 20 extending through perfusion lumen 130.

An inflation tube 20 extending through perfusion lumen 130 can be attached to one or more of the inner sides(s) 106 of the toroid balloon(s) 102, optionally via side port(s) 23, such that as perfusion lumen diameter Dp increases during inflation, the portion of any inflation tube 20 attached to balloon(s) 102 can move radially outward with the movement of the corresponding inner side(s) 106 of balloon wall(s) 104, father away from longitudinal axis Ax, and optionally away from nosecone shaft 26, as shown in FIG. 2B for example. In some examples, this radial displacement of the distal portion of the inflation tube 20 can form a bent or deflected region of the inflation tube 20, banding from a portion of the tube 20 situated closer to central axis Ax, for example along tube portions that extend through a shaft of the delivery apparatus, such as delivery shaft 14 and/or balloon assembly shaft 18, to the greater diameter at the tube's connected point to balloon assembly 100.

The toroid balloons 102 of balloon assembly 100 may be configured to be in a deflated or undeployed state (see FIG. 2A) for being positioned in a lumen of a delivery apparatus 10, and may be configured to be inflated to a deployed/expanded/inflated state as shown in FIGS. 2B-2C. The tube lumen(s) 22 may be utilized to inflate the toroid balloons 102 to transition the balloon assembly 100 to an inflated or deployed state, and may be utilized to deflate the toroid balloon 102 to transition the balloon assembly 100 to a deflated or undeployed state. Each toroid balloon 102 can be formed as a unitary component comprising a single cavity 114. Alternatively, balloon assembly 100 can be formed as a unitary component in which a plurality of cavities 114 are separated from each other.

Nosecone shaft 26, inflation tube(s) 20, optional balloon assembly shaft 18, and optional delivery shaft 14 of delivery apparatus 10, can be formed from any of various suitable materials, such as nylon, braided stainless steel wires, or a polyether block amide (commercially available as Pebax®). In some examples, inflation tube(s) 20, optional balloon assembly shaft 18, and optional delivery shaft 14 of delivery apparatus 10 have longitudinal sections formed from different materials in order to vary the flexibility of the shafts along their lengths. In some examples, nosecone shaft 26 has an inner liner or layer formed of Teflon® to minimize sliding friction with a guidewire 36.

The outer sides 108 of at least some of the toroid balloons 102 are configured to receive and urge against a prosthetic device 12 (i.e., to radially expand the prosthetic device, such as a prosthetic heart valve) and/or configured to urge against an inner wall of a patient lumen 40 (i.e., during a valvuloplasty procedure).

Advantageously, by providing a balloon assembly 100 defining a perfusion lumen 130 that allows continuous blood perfusion therethrough, a physician can have additional time to perform the interventional procedures, such as valvuloplasty or prosthetic device implantation that allow the physician to deploy (or collapse) the prosthetic device, while the risk of significant adverse effects due to blood occlusion through the orifice or passageway can be reduced.

Each toroid balloon 102 may be made of one polymer, or use several layers or a mix of different polymers. Polymers such as Nylon, PEBAX, PET, parylene and/or polyurethane may be used to make a toroid balloon 102. Various techniques can be utilized for manufacturing a toroid balloon 102. For example, a toroid balloon 102 may be fabricated by blow molding, wherein the mold can be shaped to provide the desired toroid shape of the balloon 102.

Each toroid balloon 102 defines, in an inflated state thereof, a toroidal outer diameter To, which is the outer overall diameter of the balloon 102 relative to longitudinal axis Ax, measured between opposite ends of the outer side 108. A plurality of toroid balloons 102 share a common longitudinal axis Ax of the balloon assembly 100, such that their toroidal outer diameters To together define the outer diameter of the balloon assembly 100 at the corresponding points along longitudinal axis Ax. The cavity of each cavity 114 of a toroid balloon 102 further defines, at a cross-section thereof, a cavity radial dimension Cr, extending between the inner side 106 and the outer side 108, and a cavity axial dimension Ca, extending between the opposite lateral sides (i.e., between the proximal side 110 and the distal side 112). In some cases, the cross-sectional shape of cavity 114 in the inflated state is circular, such that the cavity radial dimension Cr and the cavity axial dimension Ca are equal to each other, constituting a diameter of the cavity's cross-section. In some examples, the cavity 114 can define a non-circular cross-sectional shape, such as an elliptic or rectangular shape, in which case the cavity radial dimension Cr and the cavity axial dimension Ca can be different from each other.

In some examples, the plurality of toroid balloons 102 can be similarly dimensioned, as shown in FIGS. 2A-2C, meaning that the toroidal outer diameter To, the cavity radial dimension Cr and the cavity axial dimension Ca, are similar for all balloons 102. In some examples, at least two toroid balloons of the plurality of balloons 102 can be differently dimensioned from each other, as will be demonstrated in additional examples hereinbelow.

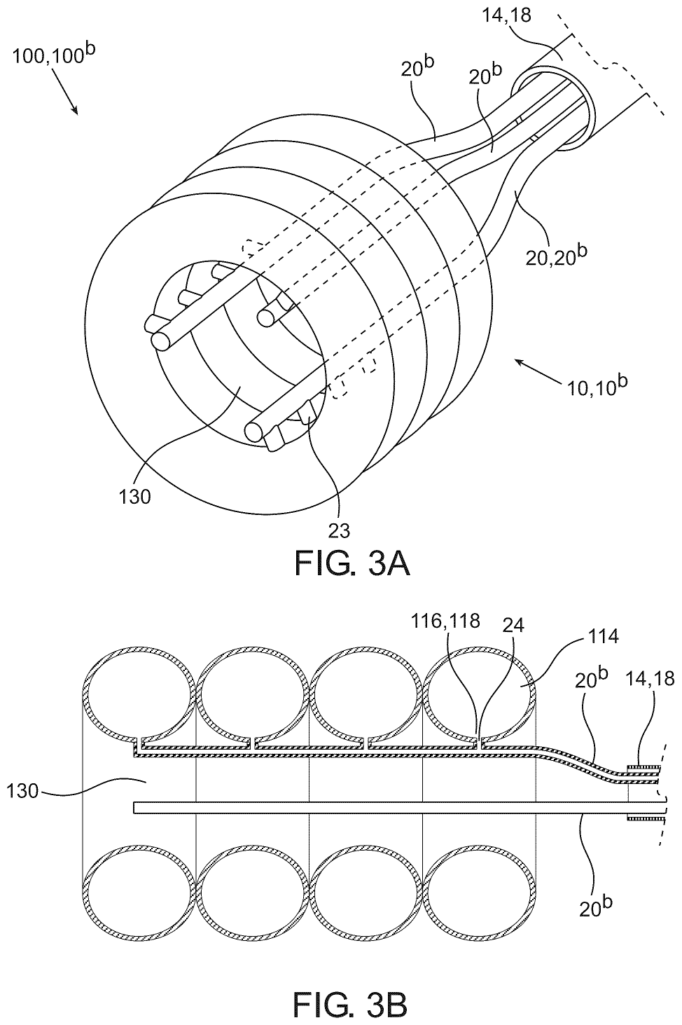

FIGS. 3A and 3B show a view in perspective and a sectional side view of a distal portion of an exemplary delivery apparatus 10^{b} that includes three inflation tubes 20^{b} extending through the perfusion lumen 130^{b} of exemplary balloon assembly 100^{b}. Delivery apparatus 10^{b} and balloon assembly 100^{b} are examples of delivery apparatus 10 and balloon assembly 100, and thus can include any of the features described for delivery apparatus 10 and balloon assembly 100 throughout the current disclosure, except that while delivery apparatus 10 can include any number of inflation tubes 20 (including a single inflation tube), delivery apparatus 10^{b} includes a plurality of inflation tubes connected to the toroid balloons. For example, delivery assembly 10^{b} can be similar to delivery assembly 10^{a} except that it includes three inflation tubes 20^{b} extending through the perfusion lumen 130^{b}. Any one of inflation tubes 20^{b} can be similar to inflation tube 20^{a} and include any of the features disclosed herein for inflation tube 20, unless otherwise stated. Similarly, balloon assembly 100^{b} can be similar to balloon assembly 100^{a}, except that its toroid balloons 102^{b} include balloon openings 116 corresponding in number to the tube openings 24 of inflation tubes 20^{b}. While three inflation tubes 20^{b} are shown in FIGS. 3A-3B, it is to be understood that any other number is contemplated, such as two inflation tubes or more than three inflation tubes.

In the illustrated example, the inflation tubes 20^{b} are disposed around the circumference of prefusion lumen 130, wherein each of the inflation tubes 20^{b} is further shown to be in fluid communication with all of the toroid balloons 102^{b} via a plurality of tube openings 24, and optionally via a matching plurality of side ports 23. This results in each of the toroid balloons 102^{b} being in fluid communication with more than one inflation tube 20, wherein the plurality of toroid balloons 102^{b} can be inflated by fluid supplied from all inflation tubes 20^{b}. Each toroid balloon 102^{b} can thus include a plurality of balloon openings 116, and more specifically, a plurality of radially oriented openings 118, angularly spaced from each other along its inner side 106 as shown in, for example, FIGS. 2B and 2C. In some examples, the inflation tubes 20^{b} and a plurality of radially oriented openings 118 of any toroid balloon 102^{b} can be evenly or unevenly angularly spaced from each other around longitudinal axis Ax.

An advantage offered by such a configuration is that each toroid balloon 102^{b} can be inflated more evenly along its circumference, due to inflation fluid being streamed into its cavity 114 from several tube opening 24 disposed around the circumference of the inner side 106. Furthermore, when a plurality of inflation tubes 20 are utilized to inflate the balloons 102, each inflation tube 20 can be formed to have a narrower tube diameter Dt. Finally, the illustrated configuration can provide better control over the rate of inflation. For example, when rapid inflation is desired, inflation fluid can be supplied via all inflation tubes 20^{b} simultaneously. In contrast, when a slower inflation rate is desired, inflation fluid can be supplied through only one or some of the inflation tubes 20^{b} feeding the respective toroid balloon(s) 102^{b}.

In some examples, a balloon assembly 100 can include a proximal section 122, a distal section 124, and an intermediate section 126 disposed between the proximal section 122 and the distal section 124, wherein each of the sections 122, 124, 126 comprises one or more of the toroid balloons 102. In some examples, at least one of the toroid balloons 102 in at least one of the sections 122, 124, 126 is differently dimensioned from at least one of the toroid balloons 102 in at least one of the other sections 122, 124, 126.

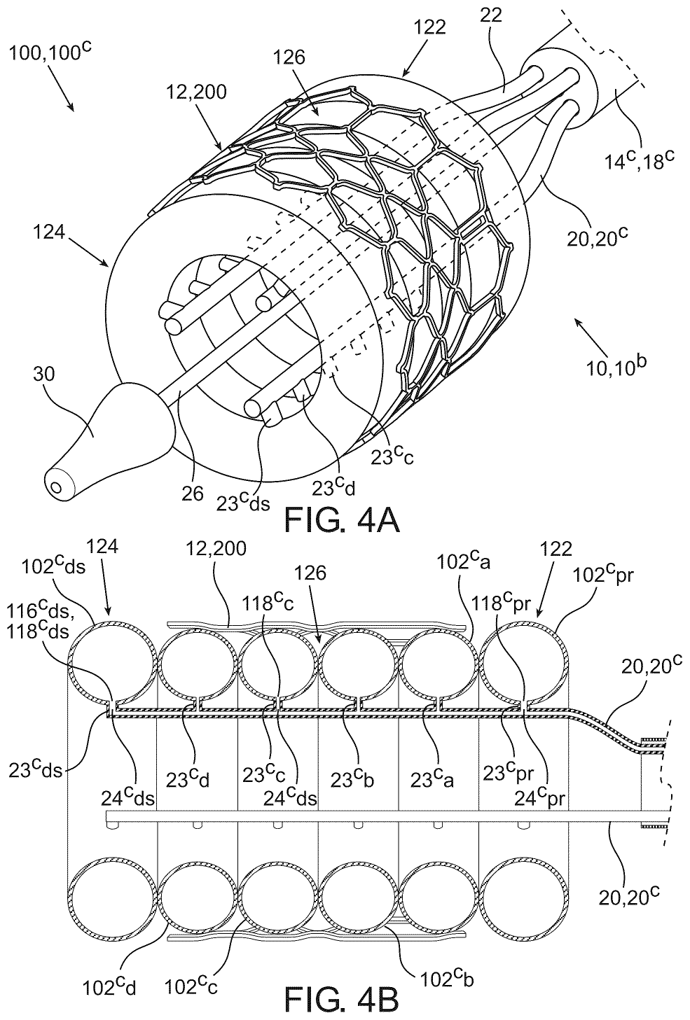

FIGS. 4A and 4B show a view in perspective and a sectional side view of a distal portion of an exemplary delivery apparatus 10^{c} that includes a balloon assembly 100^{c}. Delivery apparatus 10^{c} and balloon assembly 100^{c} are examples of delivery apparatus 10 and balloon assembly 100, and thus can include any of the features described for delivery apparatus 10 and balloon assembly 100 throughout the current disclosure, except that while delivery apparatus 10 can include a balloon assembly 100 that does or does not include proximal, intermediate and distal sections, delivery apparatus 10^{c} includes a balloon assembly 100^{c} that includes three sections, each of which including one or more toroid balloons. For example, balloon assembly 100^{c} includes a proximal section 122^{c}, a distal section 124^{c}, and an intermediate section 126^{c}. The proximal section 122^{c} includes toroid balloon 102^{c}pr, the distal section 124^{c} includes toroid balloons 102^{c}ds, and the intermediate section 126^{c} includes toroid balloons 102^{c}a, 102^{c}b, 102^{c}c, 102^{c}d, which can be similar to toroid balloons 102 any one of balloon assemblies 100^{a} and 100^{b}, or any other balloon assembly 100 or an intermediate section 126 thereof, disclosed herein. It is to be understood that one toroid balloon 102 included in each of the proximal and distal sections 122, 124, as well as four toroid balloons 102 included in the intermediate section 126, are shown by way of illustration and not limitation, and that any of the sections can include any other number of toroid balloons 102. For example, any of the proximal and distal sections 122, 124 can include two or more toroid balloons.

Three inflation tubes 20^{c} are illustrated to extend through perfusion lumen 130^{c}, each of which optionally in fluid communication with all of the toroid balloons 102^{c} of the balloon assembly 100^{c}, in a similar manner to that described above with respect to inflation tubes 20^{b}. However, it is to be understood that any other arrangement is contemplated, such as a single inflation tube as shown in FIGS. 2A-2C, or other exemplary arrangements of one or more inflations tubes 20 disclosed herein, unless otherwise stated. Nosecone 30 and nosecone shaft 26 are shown in some of the drawings (such as FIGS. 2A-2C and FIG. 4A) but removed from view in other drawings (such as FIGS. 3A-3B, 4B and 6-12B) for simplicity.

In some examples, one or more inflation tubes 20 and optionally the nosecone shaft 26 can extend through a multi-lumen shaft of the delivery apparatus 10, which can be a multi-lumen balloon assembly shaft 18^{c} or a multi-lumen delivery shaft 14^{c}, as illustrated for example in FIG. 4A. In some examples, one or more inflation tubes 20 and optionally the nosecone shaft 26 can extend through a single lumen of a shaft of the delivery apparatus 10, which can be a balloon assembly shaft 18^{a} or a delivery shaft 14^{a}, as illustrated for example in FIG. 2C.

In some examples, at least one of the toroid balloons 102 is differently dimensioned from at least one other toroid balloon 102 of balloon assembly 100. Differently dimensioned toroid balloons 102 can be utilized to define different toroidal outer diameter To at specific regions of the balloon assembly 100. In some examples, the toroidal outer dimensions To of each of a toroid balloon 102^{c}pr of the proximal section 122^{c} and a toroid balloon 102^{c}ds of the distal section 124^{c} is greater than the toroidal outer dimensions To of at least one or some of the balloons 102^{c}, and optionally of all of the balloons 102^{c}, of the intermediate section 126^{c}. The toroidal outer dimensions To of balloon 102^{c}pr of the proximal section 122^{c} and balloon 102^{c}ds of the distal section 124^{c} can be similar to each other in some example.

As mentioned above, the balloon assembly 100 can be utilized as an expansion platform for expanding a prosthetic device 12, such as a prosthetic heart valve 200, during valve implantation procedure. An exemplary prosthetic heart valve 200 is illustrated in FIGS. 4A-4B over the intermediate section 126, wherein only a frame of the prosthetic heart valve is shown for simplicity. The length of the intermediate section 126 along the longitudinal axis Ax, dictated by the number of toroid balloons 102 it includes and their cavity axial dimensions Cr, can be designed to accommodate a prosthetic device, such as a prosthetic heart valve 200, thereon. In some example, the combined cavity radial dimensions Cr of all toroid balloons 102 of an intermediate section 126 is at least as great as, and optionally greater than, the length (or height) of a prosthetic heart valve 200 (measure between its inflow and outflow ends, as will be described further hereinbelow). The height of the prosthetic valve can change between its crimped and expanded configurations, due to prosthetic heart valve foreshortening. The length of the intermediate section 126 can be set to be at least as great as the height of the prosthetic heart valve 200 in its crimped configuration, in some examples.

Though the intermediate section 126 can include a plurality of toroid balloons 102 to achieve a desired length, as illustrated, in some examples, the intermediate section 126 can include at least one elongated toroid balloon, having a cavity axial dimension Ca which is significantly greater than the cavity radial dimension Cr. For example, while the toroid balloons 102pr and 102ds of the proximal and distal sections 122, 124 can have substantially circular cross section, such that Cr is equal to or similar to Ca, an intermediate section 126 can have a single elongated toroid balloon with a cavity axial dimension Ca that corresponds to the desired length of the intermediate section, which is significantly greater than that of the balloons 102pr and 102ds (example not illustrated). Combinations of differently shaped balloons are also contemplates.

A configuration of the type illustrated in FIGS. 4A-4B, in which the toroidal outer diameters To of the proximal and distal sections 122, 124, can advantageously limit axial displacement of a prosthetic heart valve 200 disposed over the intermediate section 126, to prevent the prosthetic heart valve 200 from slipping away from the balloon assembly 100 and retain its axial position over the intermediate section 126 during inflation of the balloon assembly 100.

In some examples, one or more of the side ports 23 can be differently sized from another one or more of the side ports 23. Similarly, one or more of the tube openings 24 can be differently sized from another one or more of the tube openings 24, and one or more of the balloon openings 116 (such as radially oriented opening(s) 118) can be differently sized from another one or more of the balloon openings 116. FIGS. 4A-4B show an exemplary configuration in which the side ports 23^{c}ds and 23^{c}pr are wider than side ports 23^{c}a, 23^{c}b, 23^{c}c, 23^{c}d, the port openings 24^{c}ds and 24^{c}pr are wider than port openings 24^{c}a, 24^{c}b, 24^{c}c, 24^{c}d, and the radially extending balloon openings 118^{c}ds and 118^{c}pr are wider than balloon openings 118^{c}a, 118^{c}b, 118^{c}c, 118^{c}d. Differently sized side ports, tube openings and/or balloon openings allow different toroid balloons to be inflated at desired different rates of inflation. In some examples, toroid balloons dimensioned to include larger cavities, can be fed through wider side ports and/or tube openings so as to result in uniform inflation time for all toroid balloons of the balloon assembly.

The term “prosthetic heart valve”, as used herein, refers to any type of a balloon expandable prosthetic heart valve deliverable to a patient's target site over a catheter, which is radially expandable and compressible between a radially compressed, or crimped, configuration, and a radially expanded configuration. Thus, the prosthetic heart valve can be crimped on or retained by an implant delivery apparatus (such as delivery apparatus 10 shown in FIG. 1) in the radially compressed configuration during delivery, and then expanded to the radially expanded configuration once the prosthetic valve reaches the implantation site. A prosthetic heart valve of the current disclosure may include any prosthetic heart valve configured to be mounted within the native aortic valve, the native mitral valve, the native pulmonary valve, and the native tricuspid valve.

FIG. 5 shows a view in perspective on an exemplary prosthetic heart valve 200 that can be expanded by balloon assembly 100, illustrated in an expanded configuration. The prosthetic heart valve 200 can comprise an outflow end 201 and an inflow end 202, wherein the height of the valve is defined as the distance between the inflow and outflow ends 202, 201. In some instances, the outflow end 201 is the proximal end of the prosthetic heart valve 200, and the inflow end 202 is the distal end of the prosthetic heart valve 200. In some examples, depending for example on the delivery approach of the valve, the outflow end can be the distal end of the prosthetic heart valve, and the inflow end can be the distal end of the prosthetic heart valve. The term “outflow”, as used herein, refers to a region of the prosthetic heart valve through which the blood flows through and out of the prosthetic heart valve 200. The term “inflow”, as used herein, refers to a region of the prosthetic heart valve through which the blood flows into the prosthetic heart valve 200.

The prosthetic heart valve 200 comprises an annular frame 210 movable between a radially compressed configuration and a radially expanded configuration, and a valvular structure 260 mounted within the frame 210. The frame 210 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 210 can be crimped to a radially compressed configuration on the balloon assembly 100, and then expanded inside a patient by the balloon assembly 100.

In the example illustrated in FIG. 5, the frame 210 is an annular, stent-like structure comprising a plurality of intersecting struts 214. A strut 214 may be any elongated member or portion of the frame 210. The frame 210 can include a plurality of strut rungs that can collectively define one or more rows of cells 230. The frame 210 can have a cylindrical or substantially cylindrical shape having a constant diameter from the inflow end 202 to the outflow end 201 as shown, or the frame can vary in diameter along the height of the frame, as disclosed in U.S. Pat. No. 9,155,619, which is incorporated herein by reference.

The end portions of the struts 214 are forming apices 228 at the outflow end 201 and apices 229 at the inflow end 202. The struts 214 can intersect at additional junctions 227 formed between the outflow apices 228 and the inflow apices 229. The junctions 227 can be equally or unequally spaced apart from each other, and/or from the apices 228, 229, between the outflow end 201 and the inflow end 202.

At least some of the struts can be pivotable or bendable relative to each other, so as to permit frame expansion or compression. For example, the frame 210 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 260 can include a plurality of leaflets 262 (e.g., three leaflets), positioned at least partially within the frame 210, and configured to regulate flow of blood through the prosthetic heart valve 200 from the inflow end 202 to the outflow end 201. While three leaflets 262 arranged to collapse in a tricuspid arrangement are shown in the illustrated example, it will be clear that a prosthetic heart valve 200 can include any other number of leaflets 262. The leaflets 262 can be made from, in whole or part, biological material (e.g., pericardium), bio-compatible synthetic materials, or other such materials. Further details regarding transcatheter prosthetic heart valves, including the manner in which the valvular structures 260 can be coupled to the frame 210 of the prosthetic heart valve 200, can be found, for example, in U.S. Pat. Nos. 6,730,118, 7,393,360, 7,510,575, 7,993,394, 8,652,202, and 11,135,056, all of which are incorporated herein by reference in their entireties.

The leaflets 262 define a non-planar coaptation plane (not annotated) when free outflow edges thereof co-apt with each other to seal blood flow through the prosthetic heart valve 200. Adjacent leaflets 262 can be secured to one another to form commissures 280 of the valvular structure 260, which can be secured, directly or indirectly, to structural elements connected to the frame 210 or integrally formed as portions thereof, such as commissure posts, commissure windows, and the like. When the leaflets 262 are coupled to the frame and to each other, the lower edge of the resulting valvular structure 260 desirably has an undulating, curved scalloped shape. By forming the leaflets with this scalloped geometry, stresses on the leaflets 262 are reduced which, in turn, improves durability of the prosthetic valve. Moreover, by virtue of the scalloped shape, folds and ripples at the belly of each leaflet, which can cause early calcification in those areas, can be eliminated or at least minimized. The scalloped geometry also reduces the amount of tissue material used to form the valvular structure, thereby allowing a smaller, more even crimped profile at the inflow end of the valve.

In some examples, the prosthetic valve can further comprise at least one skirt or sealing member. FIG. 5 shows an example of a prosthetic heart valve 200 that includes an inner skirt 206, which can be secured to the inner surface of the frame 210. Such an inner skirt 206 can be configured to function, for example, as a sealing member to prevent or decrease perivalvular leakage. An inner skirt 206 can further function as an anchoring region for valvular structure 260 to the frame 210, and/or function to protect the leaflets 262 against damage which may be caused by contact with the frame 210, for example during valve crimping or during working cycles of the prosthetic heart valve 200. In some examples, the prosthetic heart valve 200 can comprise an outer skirt 207 mounted on the outer surface of frame 210, configure to function, for example, as a sealing member retained between the frame 210 and the surrounding tissue of the native annulus against which the prosthetic valve is mounted, thereby reducing risk of paravalvular leakage past the prosthetic heart valve 200.

Any of the inner skirt 206 and/or outer skirt 207 can be made of various suitable biocompatible materials, such as, but not limited to, various synthetic materials (e.g., PET) or natural tissue (e.g., pericardial tissue). In some cases, the inner skirt 206 can be formed of a single sheet of material that extends continuously around the inner surface of frame 210. In some cases, the outer skirt 207 can be formed of a single sheet of material that extends continuously around the outer surface of frame 210.

In some examples, a delivery apparatus 10 comprising a balloon assembly 100 and a prosthetic heart valve assembled thereon, can be packaged in a sterile package that can be supplied to end users for storage and eventual use. In some examples, the leaflets of the prosthetic heart valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic heart valve 200 and the delivery apparatus 10 can be free of any liquid. Methods for treating tissue leaflets for dry storage are disclosed in U.S. Pat. Nos. 8,007,992 and 8,357,387, both of which documents are incorporated herein by reference.

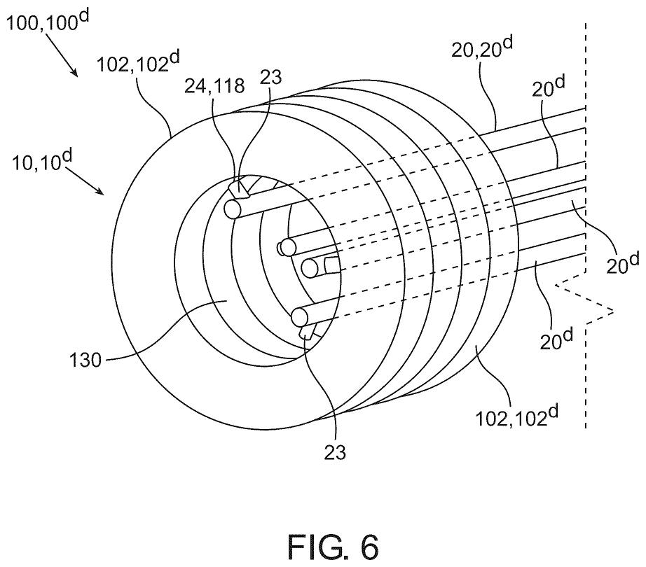

FIG. 6 shows a view in perspective of a distal portion of another exemplary delivery apparatus 10^{d} that includes a plurality of inflation tubes 20^{d} extending through perfusion lumen 130, each coupled to and being in fluid communication with a different toroid balloon 102^{d} of balloon assembly 100^{d}. Delivery apparatus 10^{d} and balloon assembly 100^{d} are examples of delivery apparatus 10 and balloon assembly 100, and thus can include any of the features described for delivery apparatus 10 and balloon assembly 100 throughout the current disclosure, except that while delivery apparatus 10 can include any number of inflation tubes 20, any of which optionally being in fluid communication with some and/or all of the toroid balloons 102, delivery apparatus 10^{d} includes a plurality of inflation tubes, at least two of which are in fluid communications with a separate corresponding one of the toroid balloons 102^{d}. For example, balloon assembly 100^{d} of delivery apparatus 10^{d} can be similar to balloon assemblies 100^{a} and/or 100^{b}, except that each toroid balloon 102^{d} is illustrated to include a single radially oriented opening 118. The total number of separate inflation tubes 20^{d} can correspond to the number of toroid balloons 102^{d}, such as four inflation tubes 20^{d} that can be coupled to the four toroid balloons 102^{d} in the illustrated example, with each inflation tube 20^{d} including a single tube opening 24, optionally at an end of a side port 23, aligned with and in communication with the radially oriented opening 118 of the corresponding toroid balloon 102^{d}.

This configuration allows each inflation tube 20^{d} to separately inflate a corresponding one of the toroid balloons 102^{d}. Since the tube opening 24 of each inflation tube 20^{d} is aligned with a corresponding axially oriented opening 120 of a different toroid balloon 102^{d} disposed at a different axial position with respect to the other toroid balloons 102^{d}, each inflation tube 20^{d} can have a different length, terminating at a distal end thereof that corresponds to the axial position of its tube opening 24 and/or tube port 23. As mentioned above, different inflation tubes 20 can be independently fed by inflation fluid, for example by connecting them to different syringes or to different outlets of a pump. This configuration provide improved control over the inflation procedure, allowing each of the toroid balloons to be independently inflated through a respective inflation tube coupled thereto. For example, one or more of the toroid balloons 102 can be inflated at a rate which is greater than that of one or more other toroid balloons, or one or more of the toroid balloons 102 can be less or more inflated, to result in a greater or narrower toroidal outer diameter To, than that of one or more other toroid balloons.

In some cases, it may be desirable to expand the prosthetic heart valve 200 to a deployed shape having a non-uniform diameter. The deployed shape of the prosthetic heart valve 200 is important because it can affect how the prosthetic heart valve anchors or interfaces with the native valve annulus, the proportions of the prosthetic heart valve 200 that are located in one chamber versus the other chamber across the native valve, and/or the location(s) where the prosthetic heart valve engages the native tissue. The shape of the expanded prosthetic heart valve can also affect various hemodynamic parameters of the prosthetic heart valve, such as the pressure drop across the prosthetic heart valve, the orifice area at the inflow and outflow ends, and the degree to which the leaflets open and close during valve operation.

Selective inflation of the toroid balloons 102 to different toroidal outer diameter To can be configured to expand the prosthetic heart valve 200 to a desired shape, such as a Y-shape, an hourglass shape, a V-shape, an A-shape or frustoconical shape, and the like. For example, to achieve a V-shaped expansion, with the inflow end 202 of the prosthetic heart valve being narrower than the outflow end 201, a greater volume of inflation fluid can be supplied to proximal toroid balloon(s) 102, resulting in a greater toroidal outer diameter To of the more proximal toroid balloon(s) 102 compared to more distal toroid balloon(s) 102, urging the outflow end 201 of the prosthetic heart valve 200 to expand to a greater diameter, relative to the inflow end 202.

Another advantage of the proposed configuration is its flexibility with respect to various types and/or sizes of prosthetic devices it can be utilized with. For example, prosthetic valves can be provided in different nominal sizes, each nominal size associated with a different range of diameters the valve can be expended to. A single balloon assembly 100 can be used in combination with various sizes of such prosthetic heart valves, for example by inflating all or most of the toroid balloons 102 when used in combination with a large or relatively elongated prosthetic heart valve, while a smaller number of balloons 102^{d} can be inflated to expand a smaller or shorter valve carried thereon, leaving the rest of the toroid balloons 102 optionally deflated.

While the inflation tubes 20^{d} are illustrated in FIG. 6 in combination with a balloon assembly 100 that includes a plurality of similarly sized toroid balloons 102, it is to be understood that the arrangement described above for inflation tubes 20^{d} can be similarly coupled to any other exemplary balloon assembly 100 disclosed herein, including a balloon assembly that includes a proximal sections 122, distal section 124, and intermediate section 126, as described above with respect to balloon assembly 100^{c}. Moreover, it is to be understood that a delivery assembly 10 can include more than one type inflation tube, including at least one inflation tube which can be similar to an inflation tube 20^{d} described above, which includes a single tube opening 24 in fluid communication with a corresponding toroid balloon 102, combined with at least one inflation tube which can be similar to an inflation tube 20^{a} described above, which includes a plurality of tube openings 24 optionally in fluid communication with all of the toroid balloons 102.

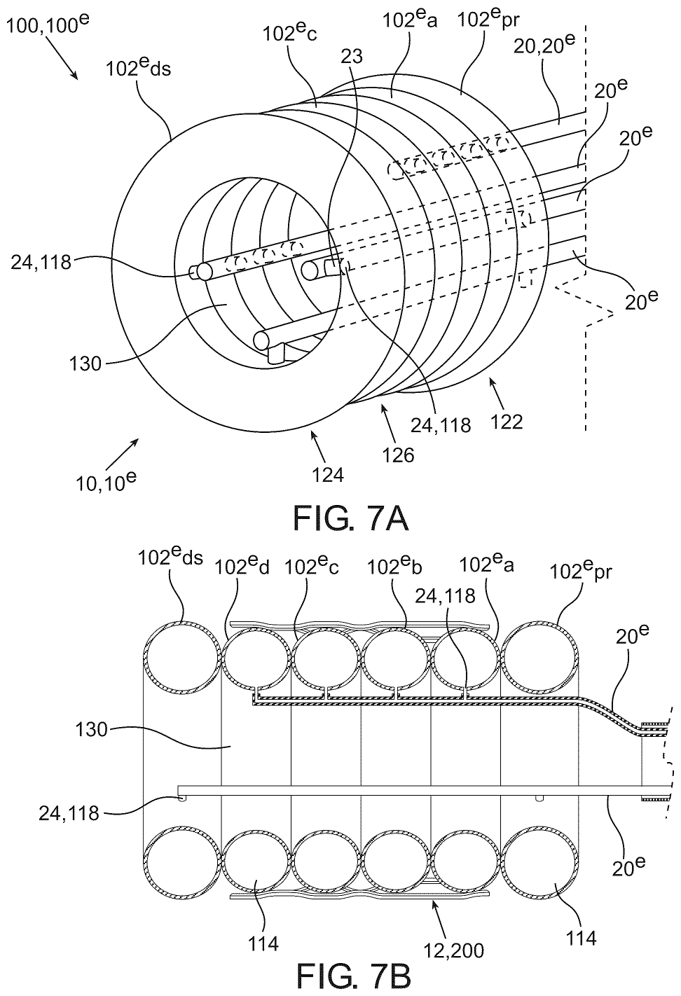

FIGS. 7A and 7B show a view in perspective and a sectional side view of a distal portion of another exemplary delivery apparatus 10^{e} including a balloon assembly 100^{e}. Delivery apparatus 10^{e} and balloon assembly 100^{e} are examples of delivery apparatus 10 and balloon assembly 100, and thus can include any of the features described for delivery apparatus 10 and balloon assembly 100 throughout the current disclosure, except that while delivery apparatus 10 can include any number of inflation tubes 20, any of which optionally being in fluid communication with one and/or all of the toroid balloons 102, delivery apparatus 10^{e} includes a plurality of inflation tubes, at least one of which is in fluid communications with some, but not necessarily all, of the toroid balloons 102^{e}. For example, delivery apparatus 10^{e} can be similar to delivery apparatus 10^{c} and/or delivery apparatus 10^{d}, except that delivery apparatus 10^{e} includes a plurality of inflation tubes 20^{e} extending through perfusion lumen 130, wherein at least one of the inflation tubes 20^{e} is in fluid communication with more than one toroid balloon 102^{e} of balloon assembly 100^{e}, but not necessarily with all toroid balloon 102^{e}. Balloon assembly 100^{e} can be similar to balloon assembly 100^{c} as shown in the example illustrated in FIGS. 7A-7B, as well as to balloon assembly 100^{a}.

In the illustrated example, four inflation tubes 20^{e} are shown to extend through perfusion lumen 130 (only two of which are visible in the sectional view of FIG. 7B), wherein two of the inflation tubes 20^{e} can be similarly constructed to include a plurality of tube openings 24, together feeding all of the toroid balloons 102^{e} of the intermediate section, while the two other inflation tubes 20^{e} can each include two tube openings 24, connected to and in fluid communication with the cavities 114 of the toroid balloon 102^{e}pr of the proximal section 122 and the toroid balloon 102^{e}ds of the distal section 124. This configuration is shown by way of illustration and not limitation, wherein: (a) an inflation tube 20 can be in fluid communication with two or more toroid balloons; (b) two or more inflation tubes 20 can be in fluid communication with a plurality of toroid balloons 102, with at least one of the toroid balloons 102 being in fluid communication of tube openings 24 of both (or more than two) inflation tubes 20; and (c) any combination of (a) and (b), optionally with one or more additional inflation tube 20 that can be in fluid communication with a single one of the toroid balloons 102.

Different toroid balloons 102 of the same balloon assembly 100 can have different numbers of balloon openings 116. While each toroid balloon 102^{e} in the example illustrated in FIGS. 7A-7B can include two balloon openings 116, in some examples, all of the balloons 102^{e} of the intermediate section 126 can have two radially oriented openings 116 if each of these balloons is fed by two inflation tubes 20^{e} as in the illustrated example, while each of the toroid balloon 102^{e}pr of the proximal section 122 and the toroid balloon 102^{e}ds of the distal section 124 can alternatively include a single radially oriented opening 118, for example when these balloon are fed by a single inflation tube 20.

Various arrangements of subsets of the toroid balloon(s) 102, each subset defined as including either a single toroid balloon 102 or a plurality of toroid balloons 102, can be in fluid communication with common or separate inflation tubes 20, designed to allow better control over the inflation volume injected into each subset of toroid balloon(s) 102, utilized generally to achieve the same goals as described above with respect to FIGS. 6A-6B, such as expanding a prosthetic heart valve 200 to a desired shape or inflating only a portion of balloons according to the size of the prosthetic heart valve 200 used in combination with the balloon assembly 100, with the main difference being in that it may be possible to control the inflation of subsets that include several toroid balloons 102, instead of individually controlling each one of the toroid balloons.

In some examples, the intermediate section 126 includes at least one toroid balloon 102 differently dimensioned that at least one other toroid balloon 102 of the intermediate section 126. For example, toroid balloons 102^{e}b and 102^{e}c at the center of intermediate section 126 are illustrated in FIGS. 7A-7B to have a toroidal outer diameter To which is less than the diameter To of toroid balloons 102^{e}a and 102^{e}d at the proximal and distal end portions of the intermediate section 126. Such a configuration can urge a prosthetic heart valve 200 expanded thereby, to assume an hourglass configuration, barrower at the mid-portion of the valve between its inflow and outflow ends. Thus, differently sized toroidal balloons 102 can be utilized to expand the prosthetic heart valve 200 to a desired shape, such as a Y-shape, an hourglass shape, a V-shape, an A-shape or frustoconical shape, and the like. Controlling the expanded shape of a prosthetic heart valve by differently sized toroid balloons 102 can be performed while all (or at least some of) the toroid balloons 102 are fed from one or more common inflation tube(s) 20, or even from more than one inflation tube(s) 20 that can be supplied by the same fluid reservoir, without needing to separately feed each or some of the balloons with different fluid volumes, eventually achieving a similar goal. Nevertheless, both differently sized toroid balloons 102 and controlling the volume of inflation fluid streamed to each or some of the balloons can be used in combination, in some examples.

While the exemplary delivery apparatus 10^{e} illustrated in FIGS. 7A-7B shows an arrangement of inflation tubes 20 in fluid communications with subsets of toroid balloons, and a balloon assembly 100 having differently sized toroid balloons 102, it is to be understood that these are two different optional configurations illustrated in the same drawings for convenience, and that the fact that both are illustrated together is not meant to be interpreted to necessitate both in the same delivery apparatus 10. In some examples, a delivery apparatus 10 can include differently sized toroid balloons 102 at any section of the balloon assembly, including the intermediate section 126. In some examples, a delivery apparatus 10 can include one or more inflation tube(s) 20 in fluid communication with subsets of toroid balloon(s) 102.

While the example illustrated in FIGS. 7A-7B shows a specific arrangement of a delivery assembly 10^{e} that includes a plurality of inflation tubes 20^{e}, each of which is in fluid communication with some but not all of the toroid balloons 102^{e}, it is to be understood that this is shown by way of illustration and not limitation, and that features exemplified with respect to FIGS. 7A-7B can be combined with features of any other exemplary device and/or assembly disclosed herein. For example, a delivery assembly 10 can include a plurality of inflation tubes 20, at least one of which includes a single tube opening 24 in fluid communication with a corresponding one of the toroid balloons 102, in a similar manner to that described with respect to inflation tube 20^{d}, and at least another one which includes a plurality of tube openings 24 that can be coupled to some of the toroid balloons 102, in a similar manner to that described with respect to inflation tube 20^{e}, and/or to all toroid balloons 102, in a similar manner to that described with respect to inflation tube 20^{a} or 20^{b}.

When a balloon assembly 100 is used in combination with a prosthetic heart valve 200 for prosthetic heart valve implantation procedures, it can advantageously allow free unblocked flow through perfusion lumen 130 while inflated, but such flow may be unregulated. For example, flow through a native heart valve is controlled by the native leaflets, configured to prevent backflow when the leaflets are closed. For example, a native aortic valve regulates blood flow therethrough, allowing blood flow from the left ventricle into the aorta during the systolic phase, and preventing backflow into the left ventricle during the diastolic phase. Once a prosthetic heart valve 200 is implanted in position and the balloon assembly 100 is retrieved, the valvular structure 260 of the prosthetic heart valve 200 is designed to control flow through the valve 200 in a similar manner. However, during the implantation procedure, while the balloon assembly 100 is inflated and the prosthetic heart valve 200 is not yet functional, blood can flow through the perfusion lumen 130 of the balloon assembly 100 in any direction, including back from the aorta into the left ventricle when inflated in a native aortic valve for example, irrespective of systolic or diastolic phases. Thus, while blood perfusion is not completely blocked by a balloon assembly 100, when compared with conventional inflatable balloons, the duration of the procedure can be still time limited due to such risks of uncontrolled blood flow through the prefusion lumen.

In some examples, a balloon assembly 100 further comprises a flexible valve 150 mounted in the balloon assembly, for example attached to a portion of the balloon wall 104, such as an inner side 106 of wall 104, of at least one of the toroid balloons 102, wherein the flexible valve 150 is configured to regulate flow (such as blood flow) through the perfusion lumen 130 in the inflated state. A flexible valve 150 can serve, in some examples, as a temporary flexible valve, for regulating blood flow through the perfusion lumen 130 during a valvuloplasty or prosthetic device implantation procedure.

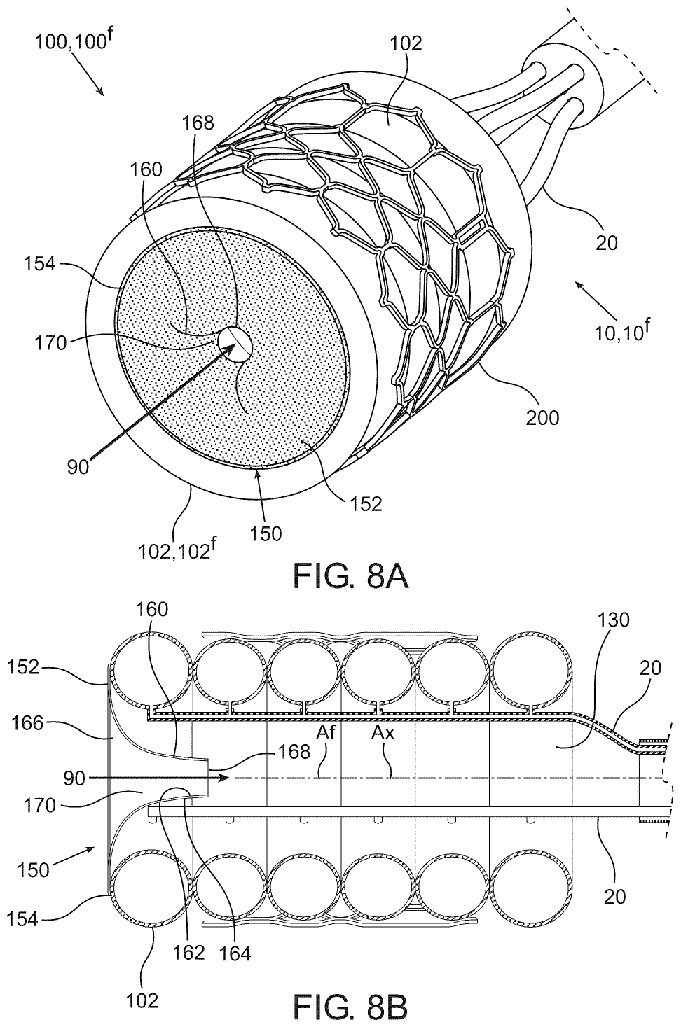

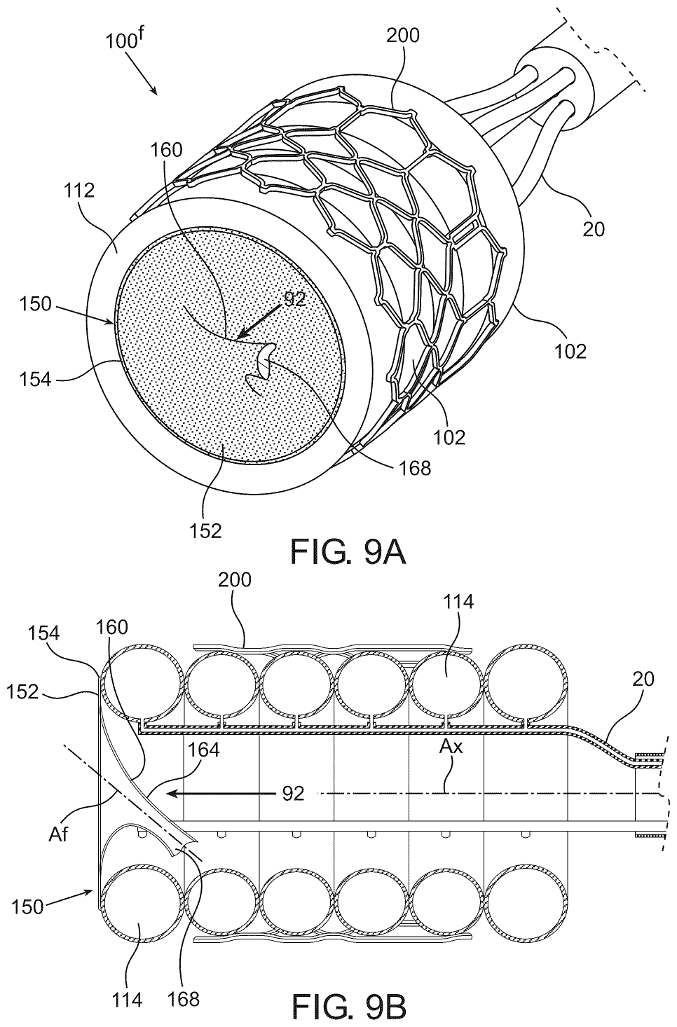

FIGS. 8A and 8B show a view in perspective and a sectional side view of a distal portion of a delivery apparatus 10^{f} with an exemplary balloon assembly 100^{f}. Delivery apparatus 10^{f} and balloon assembly 100^{f} are examples of delivery apparatus 10 and balloon assembly 100, and thus can include any of the features described for delivery apparatus 10 and balloon assembly 100 throughout the current disclosure, except that while delivery apparatus 10 can include a balloon assembly 100 defining a perfusion lumen 130 that can optionally allow blood flow therethrough in the inflated state, delivery apparatus 10^{f} further includes a flexible valve 150 configured to regulate flow through the perfusion lumen. For example, balloon assembly 100^{f} can be similar to any of balloon assemblies 100^{a}, 100^{b}, 100^{c}, 100^{d} and/or 100^{e}, and delivery assembly 10 can be similar to any of delivery assemblies 10^{a}, 10^{b}, 10^{c}, 10^{d} and/or 10^{e}, except that delivery assembly 10^{f} further comprises a flexible valve 150 attached to at least one of the toroid balloons 102^{f} of balloon assembly 100^{f}. The flexible valve 150 is shown in an open state in FIGS. 8A and 8B. FIGS. 9A and 9B show a view in perspective and a sectional side view of the delivery apparatus 10^{f} with balloon assembly 100^{f} of FIG. 8A-8B, showing the flexible valve 150 in a closed state. The flexible valve 150 comprises a valve base 152 attached at an outer circumference 154 thereof to at least one toroid balloon 102, and a flexible funnel 160 extending proximally from the valve base 152 to an open-ended funnel outlet 168. A funnel inlet 166, opposite to funnel outlet 168, is defined at the plane of the valve base 152, such that a channel 170 extends along a funnel axis Af extending from the funnel inlet 166 to the funnel outlet 168.

The valve base 152 can be formed, in some examples, of a relatively flexible membrane. The flexible funnel 160 can, in some examples, continuously extend from the valve base 152, such that the valve base 152 and the flexible funnel 160 are integrally formed to form a unitary components. The flexible funnel 160 further defines a funnel inner surface 162 facing the funnel axis Af and defining the channel 170, and an opposite funnel outer surface 164 facing the inner side 106 of toroid balloons 102. The shape of the flexible funnel 160 can transition from a wider opening at the funnel inlet 166 to a narrower opening at the funnel outlet 168, for example forming a tapering arcuate neck portion of the funnel 160 extending from the valve base 152, as illustrated.

The flexible funnel 160 is configured to bend relative to the plane of the valve base 152, which is a plane orthogonal to the longitudinal axis Ax of the balloon assembly 100, and more specifically, of the perfusion lumen 130. When blood flows in the proximal direction 90, for example, from the left ventricle toward the aortic arch during systole, when balloon assembly 100 is inflated in a native aortic valve, the flexible funnel assumes a straightened configuration, wherein the funnel axis Af is aligned with the longitudinal axis Ax, permitting blood flow through its channels 170 as illustrated in FIGS. 8A-8B. In contrast, when blood flow strives to flow in the opposite distal direction 92, such as back into the left ventricle during diastole when positioned in a native aortic valve, the flow impinges against the funnel outer surface 164, due to the tapered shape of the funnel 160, urging the flexible funnel 160 to bend sideways (for example, toward inner side 106 of toroid balloons 102 and/or toward valve base 152), such that the funnel axis Af is no longer aligned with, but is rather angled relative to the longitudinal axis Ax, effectively closing the valve 150 since the funnel outlet 168 is no longer aligned with the direction of blood flow and cannot permit backflow through channel 170. It is to be understood that in the open state, the funnel axis Af can be somewhat angled relative to longitudinal axis Ax, to an extent that still permits blood flow through the channel 170, and deflect angularly further relative to Ax in response to proximally-directed flow, to an extent sufficient to transition the flexible valve 150 to the closed state, preventing backflow through the channel 170.

The addition of a flexible valve 150 allows the balloon assembly 100 to regulate blood flow through the perfusion lumen 130 in the inflated state of the toroid balloons 102, thereby enabling the implantation procedure to be performed over a longer time period, while reducing risks associated with wither complete blocking of blood flow, or allowing unregulated blood flow throughout the inflation and implantation procedure.

The flexible valve 150 is attached along the outer circumference 154 to at least one of the toroid balloons 102 in a sealed manner, to restrict blood flow only through the channel 170. Various attachment configurations can be employed. In one example, the edge of the outer circumference 154 is attached to an inner side 106 of one of the toroid balloons 102, such as by gluing, welding, suturing, and the like. In some examples, an outermost elongated portion of the valve base 152 can axially extend along the outer circumference 154 in an axial direction, optionally attached to the inner side 106 of more than one toroid balloon 102. In some examples, the a portion of the outer circumference 154 of valve base 152 can be partially or fully wrapped around a toroid balloon 102.

The flexible valve 150 can be mounted in balloon assembly 100 at any suitable position along the longitudinal axis Ax. In the example shown for balloon assembly 100 in FIGS. 8A-9B, the flexible valve 150 is illustrated to be mounted at a distal end portion of the balloon assembly, such as by being attached to a distal-most toroid balloon 102^{e}ds. This position of attachment can be preferable for a delivery apparatus 10 that include one or more inflation tube(s) 20 extending through the perfusion lumen 130. In such cases, attaching the flexible valve 150 to a distal portion of the balloon assembly 100, which is distal to the inflation tube(s) 20, can avoid interference of the inflation tube(s) 20 with the valve in the perfusion lumen 130. Nevertheless, in some examples, the flexible valve 150 can be mounted in a position through which one or more inflation tube(s) 20 extends, with the inflation tube(s) 20 passing through the valve base 152 in a sealed manner that prevent blood flow through the valve base 152 around the passage of the inflation tube(s) 20.

When a nosecone shaft 26 or any other shaft, extends through the perfusion lumen 130, it can pass through an opening at the valve base 152 (configuration not shown), but preferably not through the channel 170, such that it would not interfere with the flexible funnel's 160 ability to bend sideways in response to backflow. The opening in the valve base through which a nosecone shaft can pass, can be sealed around the nosecone shaft 26 to prevent regurgitation during backflow (for example, in diastole). However, in some examples, complete sealing is not necessarily required, allowing for a limited amount of backflow which will still provide sufficient flow regulation functionality of the flexible valve 150 during utilization thereof.

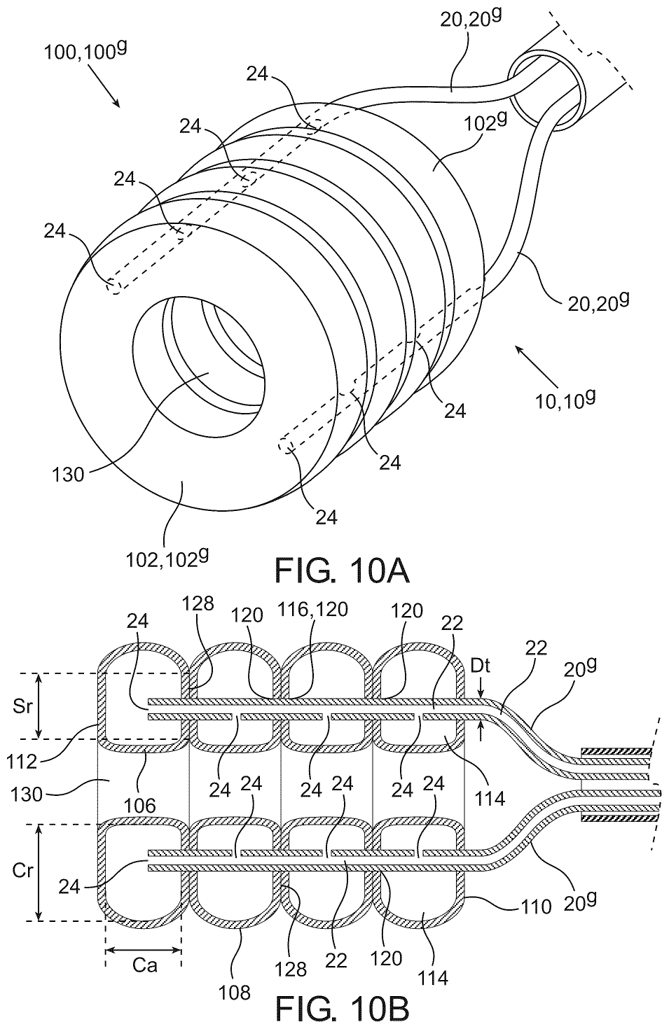

In some examples, a delivery apparatus 10 can include one or more inflation tube(s) 20 that extend through one or more of the toroid balloon(s) 102, and more specifically, through cavities 114 thereof, instead of, or in addition to, inflation tube(s) 20 that extend through perfusion lumen 130. The balloon opening(s) 116 of at least one of the toroid balloons 102 can be axially oriented opening(s) 120 formed along at least one of the lateral sides, such as proximal 110 and/or distal 112 sides, through which such inflation tube(s) 20 can extend. FIGS. 10A and 10B show a view in perspective and a sectional side view of a distal portion of an exemplary delivery apparatus 10^{g} that includes a plurality of inflation tubes 20^{g} extending through toroid balloons 102^{g} of balloon assembly 1009. Delivery apparatus 10 and balloon assembly 100^{g} are examples of delivery apparatus 10 and balloon assembly 100, and thus can include any of the features described for delivery apparatus 10 and balloon assembly 100 throughout the current disclosure, except that while delivery apparatus 10 can include inflation tubes that can optionally extend through perfusion lumen 130, delivery apparatus 10^{g} includes one or more inflation tube(s) 20^{g} extending through one or more toroid balloons 102^{g} of balloon assembly 100^{g}, wherein at least one of the inflation tube(s) 20^{g} can be in fluid communication with all toroid balloon 102^{g}. Balloon assembly 100^{g} can be similar to balloon assembly 100^{a} as shown in the example illustrated in FIGS. 10A-10B, or to any of balloon assemblies 100^{b}, 100^{c}, 100^{d} and/or 100^{e}, except that that at least one of the toroid balloons 102^{g} of balloon assembly 100^{g} includes at least one axially oriented opening 120.

Four toroid balloons 102^{g} are illustrated for exemplary balloon assembly 100^{g} by way of illustration and not limitation, and it is to be understood that any other number of toroid balloons 102^{g} is contemplated. In the illustrated example, two inflation tubes 20^{g} are illustrated, passing through all four toroid balloons 102^{g}. However, it is to be understood that a single inflation tube 20^{g} or more than two inflation tubes 20^{g} can similarly extend through any of the toroid balloons 102. Any of the inflation tubes 20 can extend through one or more of the toroid balloons 102^{g} in parallel to the longitudinal axis Ax, and to each other.

As mentioned above, various cross-sectional shapes are contemplated for toroid balloons 102. While toroid balloons 102^{a}, for example, are illustrated as donut-shaped balloons having a substantially circular cross-sectional shape, the toroid balloons 102^{g} are illustrated in FIGS. 10A-10B to be disc-shaped, having a rectangular cross-sectional, optionally with rounded corners. In the illustrated example, the cavity radial dimension Cr of toroid balloon 102^{g} is greater than the cavity axial dimension Ca. In some examples, Cr can be at least two times greater than Ca. In some examples, Cr can be similar to Ca, or Ca can be greater than Cr. It is to be understood that a specific shape, in which the outer side 108 of balloon wall 104 is more rounded than the inner side 106, is shown merely by way of illustration, and that other shapes and/or curvatures of any side of the balloon wall is contemplated.

The illustrated shape can advantageously result in a greater area of contact between adjacent balloons, to provide sufficient surface area through which inflation tube(s) 20 can pass. In some examples, the contact surface 128 between adjacent toroid balloons 102^{g} extends in the radial direction a length Sr greater than the diameter of axially oriented opening(s) 120, and/or greater than tube diameter Dt.

An inflation tube 209 extending through a toroid balloon 1029 can include a tube opening 24 exposed to, and in fluid communication with, the cavity 114 of the toroid balloon. An inflation tube 20^{g} passing through a plurality of toroid balloons 102^{g} can include a plurality of tube openings 24, exposed to and in fluid communication with some or all of the cavities 114 of the toroid balloons 102^{g} the tube extends through. In the illustrated example, each of the two inflation tubes 20^{g} can include four corresponding tube openings 24, exposed to each of the four cavities 114, such that each toroid balloon 102^{g} can be simultaneously inflated by both inflation tubes 20^{g}. In some examples, an inflation tube 20 can pass only through some of the toroid balloons 102. In some examples, an inflation tube 20 can pass through a plurality of toroid balloons 102, but include tube openings 24 exposed to the cavities of only a portion (e.g., one or more) of the cavities 114 the tube extends through.

Any inflation tube 20 extends through axially oriented opening(s) 120 of a toroid balloon 102 in a sealed manner at the interaction between the tube 20 and the opening 120 to prevent any leakage from the cavity 114. In some example, a toroid balloon 102 includes axially oriented opening(s) 120 formed in both of its lateral sides, such that each opening 120 at the proximal side 110 is facing a similar opening 120 aligned therewith at the distal side 112 of the balloon wall 104, allowing the tube 20 to extend across the entire cavity axial dimension Ca, passing through both opposing opening 120. In some examples, the inflation tube 20 can be exposed to the cavity 114 along the entire cavity axial dimension Ca, optionally including at least one tube opening 24 between both lateral sides 110, 112 of the balloon wall 104 to create fluid communication between the tube lumen 22 and cavity 114. In some examples, a channel (not shown) can be formed by the balloon material (for example, formed as an extension of balloon wall 104) between opposite axially oriented opening 120, such that the inflation tube 20 extends through and is surrounded by the channel. In such examples, the channel can have a side opening to expose a corresponding tube opening 24 to the balloon's cavity 114.

In some examples, an axially oriented opening 120 can be formed only on one lateral side, such as the proximal side 110 of the balloon wall 104, without an opposite opening aligned therewith at the distal side 112. In such examples, an inflation tube 20 can pass through the proximal side 110 into the cavity 114 and extend along a portion of the cavity axial dimension Ca, with the tube 20 terminating inside cavity 114 proximal to the distal side 112, without passing further out of the toroid balloon 102 through the distal side 112.

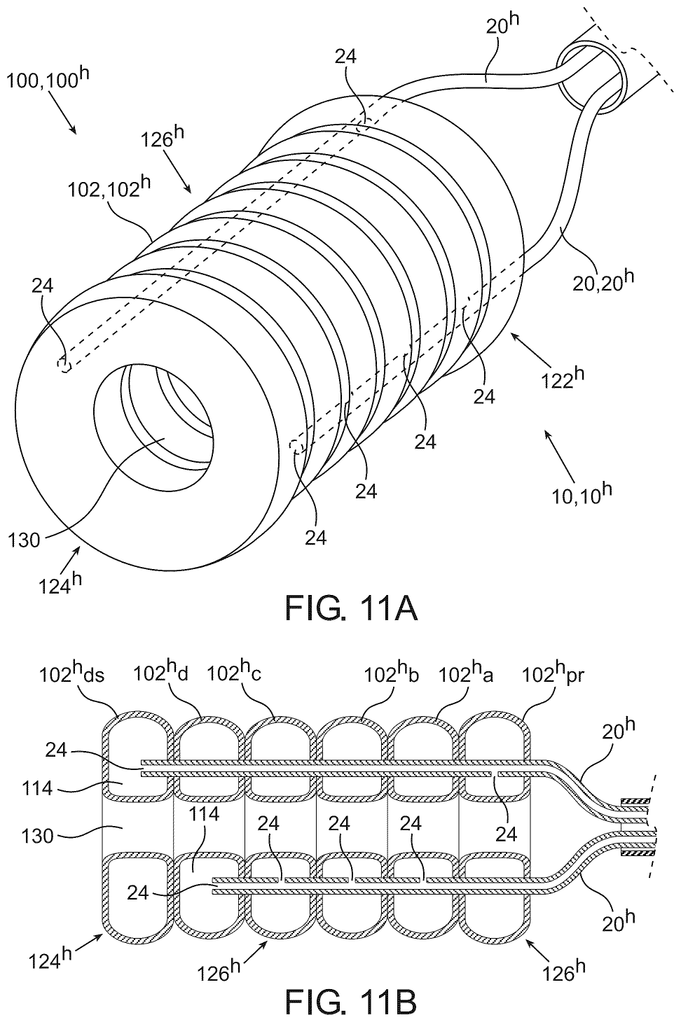

FIGS. 11A and 11B show a view in perspective and a sectional side view of a distal portion of another exemplary delivery apparatus 10^{h} that includes a plurality of inflation tubes 20^{h} extending through toroid balloons 102^{h} of balloon assembly 100^{h}. Delivery apparatus 10^{g} and balloon assembly 100^{g} are examples of delivery apparatus 10 and balloon assembly 100, and thus can include any of the features described for delivery apparatus 10 and balloon assembly 100 throughout the current disclosure, except that while delivery apparatus 10 can include inflation tubes that can optionally extend through perfusion lumen 130, delivery apparatus 10^{h} includes one or more inflation tube(s) 20^{h} extending through one or more toroid balloons 102^{g} of balloon assembly 100^{g}, wherein at least one of the inflation tube(s) 20^{h} can be in fluid communication with one or some of the toroid balloon 1029. Delivery assembly 10^{h} can be similar to delivery assembly 10^{g}, and include inflation tubes 20^{h} passing through toroid balloons 102^{h} in a similar manner described for inflation tubes 209 and toroid balloons 102^{g} described above, except that at least one inflation tube 20^{h} is in fluid communication with some (including, optionally, one), but not all, toroid balloons 102^{h}. Balloon assembly 100^{h} can be similar to balloon assembly 100^{g}, except that at least one of the toroid balloons 102^{g} can be fed by one or some, but not necessarily all, of the inflation tubes 20^{h}.

While two inflation tubes 209 are illustrated in FIGS. 10A-10B to pass through, and be in fluid communication with, all of the toroid balloons 102^{g}, the configuration of FIG. 11A-11B illustrated another arrangement of two inflation tubes 20^{h} (tough any other number is contemplated), one of which is shown to pass only through some but not all of the toroid balloons 102^{h}. For example, one inflation tube 20^{h} is shown to pass through only five of the six toroid balloons 102^{h}, and includes tube openings 24 exposed to the cavities 114 of the balloons 102^{h} of the intermediate section 126^{h}, but not to the cavities of the balloons of the proximal or distal section 122^{h} or 124^{h}, while the other inflation tube 20^{h} is provided with tube openings 24 exposed to the cavities 114 of the balloons 102^{h} of the proximal and distal section 122^{h}, 124^{h}, but not to the cavities of the balloons of the intermediate section 126^{h}. This configuration is shown by way of illustration and not limitation, wherein: (a) an inflation tube 20 extending through toroid balloons can be in fluid communication with two or more toroid balloons 102; (b) two or more inflation tubes 20 extending through toroid balloons can be in fluid communication with a plurality of toroid balloons 102, with at least one of the toroid balloons 102 being in fluid communication of tube openings 24 of both (or more than two) inflation tubes 20; and (c) any combination of (a) and (b), optionally with one or more additional inflation tube 20 that can be in fluid communication with a single one of the toroid balloons 102.

As shown in FIGS. 11A-11B, one or more of the inflation tubes 20^{h} can terminate at a position proximal to a toroid balloon not configured to be fed thereby. In the illustrated example, the inflation tube 20^{h} that feeds only the toroid balloons 102^{h} of the intermediate section 126^{h} is shown to terminate proximal to the distal section 124^{h}, without extending further into the cavity 114 of the toroid balloon 102^{h}ds of the distal section 124^{h}.

One advantage offered by inflation tubes 20 passing through toroid balloons 102 can be in a reduced crimped profiled, compared with inflation tubes 20 configured to pass through perfusion lumen 130, such that only the nosecone shaft 26 can remain within perfusion lumen 130. Another advantage relates to the flexibility offered by such arrangements in the axial position in which a flexible valve 150 can be disposed.

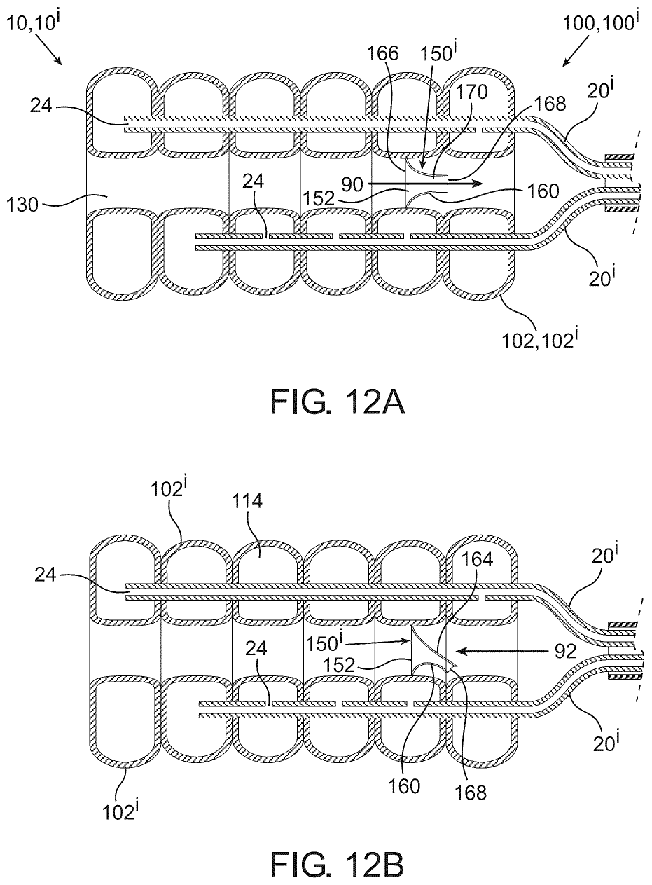

FIGS. 12A and 12B show sectional side views of a distal portion of another exemplary delivery apparatus 10ⁱ that includes inflation tubes 20ⁱ passing through toroid balloons 102ⁱ of a balloon assembly 100ⁱ that includes a flexible valve 150ⁱ, shown in open and closed states, respectively. Delivery apparatus 10^{g} and balloon assembly 100^{g} are examples of delivery apparatus 10 and balloon assembly 100, and thus can include any of the features described for delivery apparatus 10 and balloon assembly 100 throughout the current disclosure, except that the delivery apparatus necessarily 10ⁱ combines the flexible valve 150 as described above with respect to delivery apparatus 10^{f}, and inflation tube(s) extending through toroid balloons, as described above with respect to any of delivery apparatus 10^{g} and/or 10^{h}. For example, the arrangement of balloon assembly 100ⁱ and inflation tubes 20ⁱ can be similar to exemplary arrangements described above with respect to balloons assemblies 100^{h}, 100^{g} and inflation tubes 20^{h}, 20^{g}, while structural and functional aspects of the flexible valve 150ⁱ can be similar to those described above with respect to FIGS. 8A-9B. Passing inflation tube(s) 20ⁱ through toroid balloons 102ⁱ enables the flexible valve 150ⁱ to be positioned at any desired axial position within perfusion lumen 130, such as be being coupled to the proximal section 122 of a central region of the intermediate section 126, as illustrated, without being limited by, or needing to otherwise account for, inflation tube(s) 20 passing through perfusion lumen 130 as illustrated in FIGS. 8A-9B for example.

While a flexible valve 150 is described above for use with a balloon assembly that includes a plurality of toroid balloons, in some examples, a balloon assembly 100 can include a single toroid balloon 102 with a flexible valve 150 disposed therein and attached to the single balloon 102 (configuration not explicitly illustrated). The single toroid balloon 102 can be implemented according to any example disclosed herein for toroid balloons 102, in fluid communication with at least one inflation tube 20 which can be implemented according to any example disclosed herein for inflation tubes 20, mutatis mutandis.

While various examples of balloon assemblies 100 are shown throughout the drawings to include either inflation tube(s) 20 extending through the perfusion lumen 130, or inflation tube(s) 20 extending through the toroid balloon(s) 102 (for example, through cavities 114 thereof), it is to be understood that a balloon assembly can also include any combination of at least one inflation tube extending through the perfusion lumen 130, and at least one other inflation tube 20 extending through one or more of the toroid balloons 102.

While the balloon assemblies 100 are illustrated in some of the drawings (such as FIGS. 4A-4B and 7B-9B) with a prosthetic heart valve 200 mounted thereon, and in other drawings (such as FIGS. 2A-3B, 6-7A and 10A-12B), it is to be understood that this is shown merely for illustrative purpose, and that any of the balloon assemblies 100 disclosed herein can be utilized in combination with a prosthetic device 12, such as a prosthetic heart valve 200, or without a prosthetic device, for example when utilized in valvuloplasty procedures.

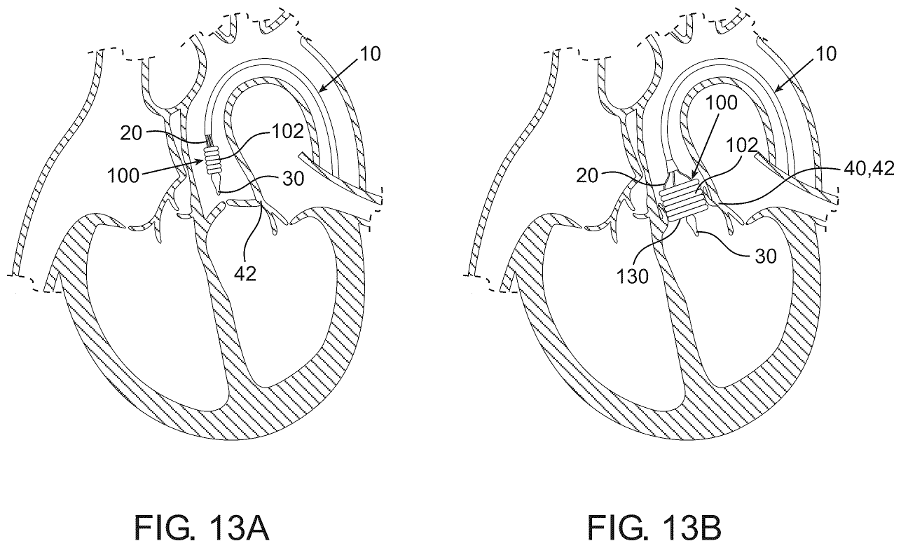

FIGS. 13A-13B illustrate an exemplary method of utilizing balloon assemblies 100 to expand an orifice or passageway of the body, such as during valvuloplasty procedures. That is, the expansion of the balloon assembly 100 can be done in the illustrated method without a prosthetic device crimped thereon in a valvuloplasty procedure. Methods disclosed herein may vary from the steps shown in FIGS. 13A-13B. FIG. 13A illustrates a step in a method of dilating a native orifice or passageway in a patient's body, such as the aortic heart valve. A delivery apparatus, such as delivery apparatus 10 illustrated in FIG. 1, may be utilized to approach the native heart valve or other orifice in the patient's body. The delivery shaft 14 may be deflected to allow the balloon assembly 100 to approach the native aortic heart valve 42 through the aortic arch. Referring to FIG. 13B, the balloon assembly 100 can be inflated to press against the native annulus and/or the native heart valve leaflets to expand the aortic annulus. While described and illustrated with respect to a native aortic heart valve, it is to be understood that balloon assemblies 100 can be similarly utilized to press against and expand other native heart valves, such as a mitral, tricuspid, or pulmonary heart valve, or any other orifice, such as stenotic portions along an artery of the patient.

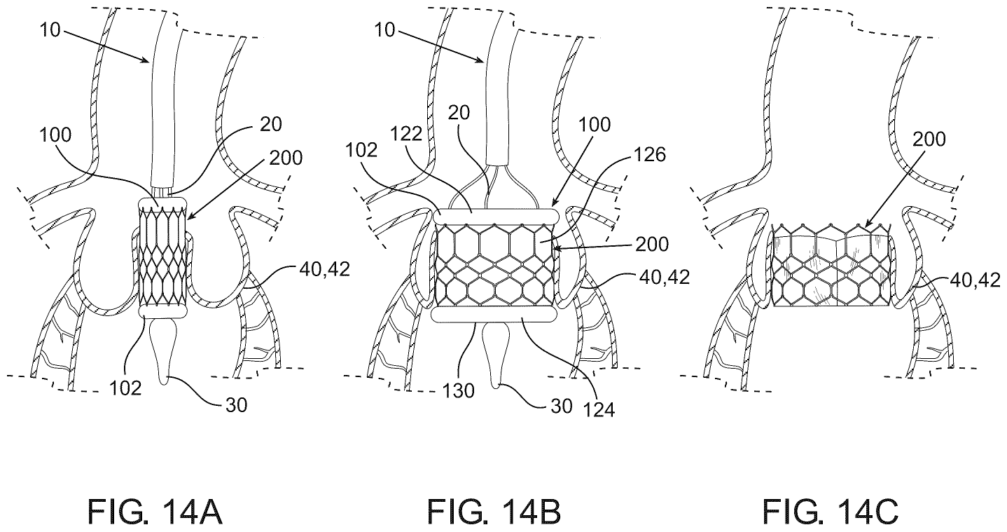

FIGS. 14A-14C illustrate a method of deploying a prosthetic heart valve 200 within a native aortic annulus 42. Referring to FIG. 14A, a delivery apparatus 10 is shown delivering a prosthetic heart valve 200 in a collapsed configuration. Delivery apparatus 10 can deliver prosthetic heart valve 200 to the treatment location using known procedures. The prosthetic heart valve can be delivered either through a transfemoral or transapical approach.

Prosthetic heart valve 200 can be mounted on balloon assembly 100. Prosthetic heart valve 200 is maneuvered within a native aortic valve annulus 42 for deployment using delivery apparatus 10. Referring to FIG. 14B, balloon assembly 100 is inflated by injecting inflation fluid 35 into the cavities 114 of toroid balloons 102 through inflation tube(s) 20. Blood is allowed to flow during systole through perfusion lumen 130, from the left ventricle toward the aorta. In some examples, when the balloon assembly 100 further comprises a flexible valve 150, blood can flow through the flexible valve 150, from the left ventricle into the aorta, during systole, while backflow during diastole is prevented as the flexible valve 150 is urged into a closed state.

After prosthetic heart valve 200 is deployed within the native aortic annulus 42, balloon assembly 100 can be deflated and removed from the aortic annulus (FIG. 14C). While a prosthetic heart valve 200 is described and illustrated in FIG. 14A-14C to expand within a native aortic heart valve, it is to be understood that prosthetic heart valve 200 can be similarly deployed in other native heart valves, such as a mitral, tricuspid, or pulmonary heart valve.

Moreover, while balloon assembly 100 is shown in combination with a prosthetic heart valve 200 in FIGS. 14A-14C, such as the prosthetic heart valve described above with respect to FIG. 5, it is to be understood that the method can be similarly utilized for deployment of other prosthetic devices by balloon assemblies 100, such as stents that can be deployed in stenotic arteries.

### Some Examples of the Disclosed Technology

Some examples of above-described technology are enumerated below. It should be noted that one feature of an example in isolation or more than one feature of the example taken in combination and, optionally, in combination with one or more features of one or more examples below are examples also falling within the disclosure of this application.

Example 1. A delivery apparatus, comprising:
a balloon assembly comprising a plurality of toroid balloons, the plurality of toroid balloons defining together a perfusion lumen along a longitudinal axis of the balloon assembly,
wherein each toroid balloon in configured to transition between a deflated state and an inflated state, and wherein each toroid balloon comprises:
a balloon wall enclosing a cavity; and
at least one balloon opening extending through a thickness of the balloon wall; and
at least one inflation tube comprising at least one tube opening which is in fluid communication with the cavity of at least one of the plurality of toroid balloons;
wherein, when the toroid balloons are in the inflated state, the perfusion lumen is configured to allow flow therethrough.

Example 2. The delivery apparatus of any example herein, particularly example 1, wherein at least one of the at least one inflation tube is in fluid communication with the cavities of more than one of the plurality of toroid balloons.

Example 3. The delivery apparatus of any example herein, particularly example 2, wherein the inflation tube which is in fluid communication with more than one cavity is in fluid communication with the cavities of all of the plurality of toroid balloons.

Example 4. The delivery apparatus of any example herein, particularly any one of examples 1 to 3, wherein the at least one inflation tube comprises a plurality of inflation tubes.

Example 5. The delivery apparatus of any example herein, particularly example 4, wherein each one of the plurality of inflation tubes is in fluid communication with the cavities of all of the plurality of toroid balloons.

Example 6. The delivery apparatus of any example herein, particularly example 4, wherein each one of the plurality of inflation tubes is in fluid communication with the cavity of a single one of the toroid balloons.

Example 7. The delivery apparatus of any example herein, particularly any one of examples 1 to 6, wherein at least one of the at least one inflation tube extends through the perfusion lumen.

Example 8. The delivery apparatus of any example herein, particularly example 7, wherein the at least one balloon opening comprises a radially oriented opening formed at an inner side of the balloon wall.

Example 9. The delivery apparatus of any example herein, particularly example 8, wherein the at least one tube opening is aligned with the corresponding balloon opening.

Example 10. The delivery apparatus of any example herein, particularly any one of examples 7 to 9, wherein the at least one inflation tube further comprises at least one side port extending from a tube lumen of the corresponding inflation tube and terminating at the corresponding tube opening.

Example 11. The delivery apparatus of any example herein, particularly any one of examples 1 to 10, wherein the plurality of toroid balloons are attached to each other.

Example 12. The delivery apparatus of any example herein, particularly any one of examples 1 to 11, wherein the perfusion lumen defines a perfusion lumen diameter which is greater in the inflated state than in the deflated state.

Example 13. The delivery apparatus of any example herein, particularly any one of examples 1 to 12, wherein each toroid balloon defines a toroidal outer diameter which is greater in the inflated state than in the deflated state.

Example 14. The delivery apparatus of any example herein, particularly example 13, wherein at least two of the toroid balloons define different toroidal outer diameters in the inflated state.

Example 15. The delivery apparatus of any example herein, particularly of example 13, wherein the balloon assembly comprises a proximal section, a distal section, and an intermediate section disposed between the proximal section and the distal section, wherein each of the sections comprises at least one of the plurality of toroid balloons, and wherein the toroidal outer diameter of at least one of the toroid balloons of the intermediate section is different from the toroidal outer diameter of at least one of the toroid balloons of the proximal section and/or of the distal section.

Example 16. The delivery apparatus of any example herein, particularly example 15, wherein the toroidal outer diameter of any one of the toroid balloons of the intermediate section is less than the toroidal outer diameter of at least one of the toroid balloons of the proximal section and/or the distal section.

Example 17. The delivery apparatus of any example herein, particularly example 15 or 16, wherein the toroidal outer diameters of the toroid balloons of the proximal section and of the distal section are identical.

Example 18. The delivery apparatus of any example herein, particularly any one of examples 15 to 17, wherein the number of toroid balloons comprised in the intermediate section is greater than the number of toroid balloons of the proximal section and/or of the distal section.

Example 19. The delivery apparatus of any example herein, particularly any one of examples 15 to 18, wherein each of the proximal section and the distal section comprises a single one of the plurality of toroid balloons.

Example 20. The delivery apparatus of any example herein, particularly any one of examples 1 to 19, wherein each of the plurality of toroid balloons defines a cavity radial dimension and a cavity axial dimension at a cross-section thereof in the inflated state, and wherein the cavity radial dimension of at least one of the plurality of toroid balloons is different than the cavity axial dimension.

Example 21. The delivery apparatus of any example herein, particularly example 20, wherein the cavity axial dimension of at least one of the toroid balloons of the intermediate section is greater than the cavity radial dimension.

Example 22. The delivery apparatus of any example herein, particularly example 20, wherein each toroid balloon defines a cavity radial dimension which is greater than a cavity axial dimension.

Example 23. The delivery apparatus of any example herein, particularly example 22, wherein the cavity radial dimension is at least two times greater than the cavity axial dimension.

Example 24. The delivery apparatus of any example herein, particularly any one of examples 1 to 23, wherein at least one of the at least one inflation tube extends through at least one of the plurality of toroid balloons.

Example 25. The delivery apparatus of any example herein, particularly example 24, wherein the at least one balloon opening comprises at least one axially oriented opening formed at a proximal side of the balloon wall.

Example 26. The delivery apparatus of any example herein, particularly example 25, wherein the at least one balloon opening further comprises at least one additional axially oriented opening formed at a distal side of the balloon wall and aligned with the corresponding axially oriented opening formed at the proximal side.

Example 27. The delivery apparatus of any example herein, particularly any one of examples 1 to 26, wherein at least one of the balloon openings is wider than at least another one of the balloon openings.

Example 28. The delivery apparatus of any example herein, particularly any one of examples 1 to 27, wherein the at least one tube opening comprises a plurality of tube openings, and wherein at least one of the plurality of tube openings is wider than at least another one of the plurality of tube openings.

Example 29. The delivery apparatus of any example herein, particularly example 10, wherein the at least one tube opening comprises a plurality of tube openings, wherein the at least one side port comprises a plurality of side ports, and wherein at least one of the plurality of side ports is wider than at least another one of the plurality of side ports.

Example 30. The delivery apparatus of any example herein, particularly any one of examples 1 to 29, further comprising a flexible valve disposed within the perfusion lumen and comprising:
a valve base attached at an outer circumference thereof to at least one of the plurality of toroid balloons; and
a flexible funnel defining a channel extending proximally from a funnel inlet at the valve base to a funnel outlet and defining a funnel axis therealong;
wherein the flexible valve is configured to transition between an open state in which proximally oriented flow can pass through the channel, and a closed state in response to distally oriented backflow such that the flow direction changes the state of the flexible valve.

Example 31. The delivery apparatus of any example herein, particularly example 30, wherein the valve base comprises a flexible membrane.

Example 32. The delivery apparatus of any example herein, particularly example 30 or 31, wherein the flexible funnel is integrally formed with the valve base.

Example 33. The delivery apparatus of any example herein, particularly any one of examples 1 to 29, wherein the funnel outlet is narrower than the funnel inlet.

Example 34. The delivery apparatus of any example herein, particularly any one of examples 1 to 29, further comprising a handle through which the at least one inflation tube extends.

Example 35. The delivery apparatus of any example herein, particularly any one of examples 1 to 34, further comprising a delivery shaft disposed around at least a portion of the at least one inflation tube, wherein the delivery shaft and the at least one inflation tube are configured to slide longitudinally relative to each other.

Example 36. The delivery apparatus of any example herein, particularly any one of examples 1 to 35, further comprising a nosecone mounted on a distal end of the delivery apparatus.

Example 37. The delivery apparatus of any example herein, particularly example 36, further comprising a nosecone shaft attached to the nosecone and extending parallel to the at least one inflation tube and through the perfusion lumen.

Example 38. The delivery apparatus of any example herein, particularly example 37, wherein the perfusion lumen is greater than an outer diameter of the nosecone shaft.

Example 39. The delivery apparatus of any example herein, particularly example 38, wherein the perfusion lumen is greater than the combined outer diameter of the nosecone shaft and outer diameters of all of the at least one inflation tubes.

Example 40. A delivery apparatus, comprising:
a balloon assembly comprising:
a toroid balloon that defines a perfusion lumen around a longitudinal axis of the balloon assembly, wherein the toroid balloon in configured to transition between a deflated state and an inflated state, and comprises:
a balloon wall enclosing a cavity; and
at least one balloon opening extending through a thickness of the balloon wall;
a flexible valve disposed within the perfusion lumen and comprising:
a valve base attached at an outer circumference thereof to the toroid balloon; and
a flexible funnel defining a channel extending proximally from a funnel inlet at the valve base to a funnel outlet and defining a funnel axis therealong; and
at least one inflation tube comprising at least one tube opening exposed to and in fluid communication with the cavity;
wherein the flexible valve is configured to transition between an open state in which proximally oriented flow can pass through the channel, and a closed state in response to distally oriented backflow such that the flow direction changes the state of the flexible valve.

Example 41. The delivery apparatus of any example herein, particularly example 40, wherein the valve base comprises a flexible membrane.

Example 42. The delivery apparatus of any example herein, particularly example 40 or 41, wherein the flexible funnel is integrally formed with the valve base.

Example 43. The delivery apparatus of any example herein, particularly any one of examples 40 to 42, wherein the funnel outlet is narrower than the funnel inlet.

Example 44. The delivery apparatus of any example herein, particularly any one of examples 40 to 43, wherein the at least one inflation tube comprises a plurality of inflation tubes.

Example 45. The delivery apparatus of any example herein, particularly any one of examples 40 to 44, wherein the at least one inflation tube extends through the perfusion lumen.

Example 46. The delivery apparatus of any example herein, particularly example 45, wherein the at least one balloon opening comprises a radially oriented.

Example 47. The delivery apparatus of any example herein, particularly example 46, wherein the at least one tube opening is aligned with the corresponding balloon opening.

Example 48. The delivery apparatus of any example herein, particularly any one of examples 45 to 47, wherein the at least one inflation tube further comprises at least one side port extending from a tube lumen of the corresponding inflation tube and terminating at the corresponding tube opening.

Example 49. The delivery apparatus of any example herein, particularly any one of examples 40 to 48, wherein the toroid balloon defines a cavity radial dimension and a cavity axial dimension at a cross-section thereof, and wherein the cavity radial dimension is different than the cavity axial dimension.

Example 50. The delivery apparatus of any example herein, particularly example 49, wherein the cavity axial is greater than the cavity radial dimension.

Example 51. The delivery apparatus of any example herein, particularly example 50, wherein the cavity axial is at least two times greater than the cavity radial dimension.

Example 52. The delivery apparatus of any example herein, particularly example 50, wherein the cavity axial is at least three times greater than the cavity radial dimension.

Example 53. The delivery apparatus of any example herein, particularly any one of examples 40 to 52, wherein at least one of the at least one inflation tube extends through the toroid balloon.

Example 54. The delivery apparatus of any example herein, particularly example 53, wherein the at least one balloon opening comprises at least one axially oriented opening formed at a proximal side of the balloon wall.

Example 55. The delivery apparatus of any example herein, particularly example 54, wherein the at least one balloon opening further comprises at least one additional axially oriented opening formed at a distal side of the balloon wall and aligned with the corresponding axially oriented opening formed at the proximal side.

Example 56. The delivery apparatus of any example herein, particularly any one of examples 40 to 55, wherein the perfusion lumen defines a perfusion lumen diameter which is greater in the inflated state than in the deflated state.

Example 57. The delivery apparatus of any example herein, particularly any one of examples 40 to 56, wherein the toroid balloon defines a toroidal outer diameter which is greater in the inflated state than in the deflated state.

Example 58. The delivery apparatus of any example herein, particularly any one of examples 40 to 57, further comprising a handle through which the at least one inflation tube extends.

Example 59. The delivery apparatus of any example herein, particularly any one of examples 40 to 58, further comprising a delivery shaft disposed around at least a portion of the at least one inflation tube, wherein the delivery shaft and the at least one inflation tube are configured to slide longitudinally relative to each other.

Example 60. The delivery apparatus of any example herein, particularly any one of examples 40 to 59, further comprising a nosecone mounted on a distal end of the delivery apparatus.

Example 61. The delivery apparatus of any example herein, particularly example 60, further comprising a nosecone shaft attached to the nosecone and extending parallel to the at least one inflation tube and through the perfusion lumen.

Example 62. A method, comprising
delivering a balloon assembly comprising a plurality of toroid balloons in a deflated state thereof, through the vasculature of a patient to a treatment site, wherein each of the toroid balloons comprises a cavity in fluid communication with at least one inflation tube; and
inflating the toroid balloons within a patient lumen and forming a perfusion lumen configured to permit blood flow therethrough;
wherein the perfusion lumen allows blood flow at least in a proximally oriented direction.

Example 63. The method of any example herein, particularly example 62, wherein inflating the toroid balloons to permit blood to flow through the perfusion lumen is performed without rapid pacing.

Example 64. The method of any example herein, particularly example 62 or 63, wherein each toroid balloon defines a toroidal outer diameter, and wherein inflating the toroid balloons comprises increasing the toroidal outer diameters such that outer sides of balloon walls of at least some of the toroid balloons press against an inner surface of the patient lumen.

Example 65. The method of any example herein, particularly example 62 or 63, wherein each toroid balloon defines a toroidal outer diameter, and wherein inflating the toroid balloons comprises increasing the toroidal outer diameters such that outer sides of balloon walls of at least some of the toroid balloons press against an inner surface of a prosthetic device disposed around the balloon, thereby expanding the prosthetic device against an inner wall of the patient lumen.

Example 66. The method of any example herein, particularly example 65, wherein the prosthetic device is a prosthetic heart valve comprising an annular frame and a valvular structure mounted within the frame, the valvular structure comprising a plurality of leaflets coupled to each other and to the frame via a plurality of commissures.

Example 67. The method of any example herein, particularly example 66, wherein the patient lumen is an annulus of a native heart valve.

Example 68. The method of any example herein, particularly any one of examples 65 to 67, wherein the prosthetic device is crimped over the balloon assembly when the balloon assembly is delivered to the treatment site.

Example 69. The method of any example herein, particularly any one of examples 65 to 67, wherein the prosthetic device is crimped over a portion of the at least one inflation tube, proximal to the balloon assembly when the balloon assembly is delivered to the treatment site.

Example 70. The method of any example herein, particularly example 69, further comprising advancing the prosthetic device onto the balloon prior to inflating the balloon.

Example 71. The method of any example herein, particularly any one of examples 62 to 70, wherein the at least one inflation tube comprises a plurality of inflation tubes.

Example 72. The method of any example herein, particularly example 71, wherein each one of the plurality of inflation tubes is in fluid communication with the cavities of all of the plurality of toroid balloons.

Example 73. The method of any example herein, particularly any one of examples 62 to 72, wherein inflating the toroid balloons comprises inflating all of the toroid balloons by simultaneously injecting inflation fluid through all of the at least one inflation tubes.

Example 74. The method of any example herein, particularly example 71, wherein at least one of the plurality of toroid balloons is in fluid communication by at least one of the inflation tubes, but is not in fluid communication with at least one other of the inflation tubes.

Example 75. The method of any example herein, particularly example 73, wherein inflating the toroid balloons is performed such that at least two of the toroid balloons are inflated sequentially.

Example 76. The method of any example herein, particularly any one of examples 62 to 75, wherein at least one of the at least one inflation tube extends through the perfusion lumen.

Example 77. The method of any example herein, particularly example 76, wherein the plurality of toroid balloons comprises radially oriented openings formed at inner sides of balloon walls thereof.

Example 78. The method of any example herein, particularly example 77, wherein the at least one inflation tube comprises one or more tube openings aligned with the corresponding radially oriented openings.

Example 79. The method of any example herein, particularly example 78, wherein the at least one inflation tube further comprises one or more side port extending from a tube lumen of the corresponding inflation tube and terminating at the corresponding tube opening.

Example 80. The method of any example herein, particularly any one of examples 62 to 79, wherein the plurality of toroid balloons are attached to each other.

Example 81. The method of any example herein, particularly any one of examples 62 to 80, wherein the perfusion lumen defines a perfusion lumen diameter, and wherein inflating the toroid balloons comprises increasing the perfusion lumen diameter.

Example 82. The method of any example herein, particularly any one of examples 64 or 65, wherein the balloon assembly comprises a proximal section, a distal section, and an intermediate section disposed between the proximal section and the distal section, wherein each of the sections comprises at least one of the plurality of toroid balloons, and wherein the toroidal outer diameter of at least one of the toroid balloons of the intermediate section is different from the toroidal outer diameter of at least one of the toroid balloons of the proximal section and/or of the distal section.

Example 83. The method of any example herein, particularly example 82, wherein the toroidal outer diameter of any one of the toroid balloon of the intermediate section is less than the toroidal outer diameter of at least one of the toroid balloons of the proximal section and/or of the distal section.

Example 84. The method of any example herein, particularly example 82 or 83, wherein the toroidal outer diameters of the toroid balloons of the proximal section and of the distal section are identical.

Example 85. The method of any example herein, particularly any one of examples 82 to 84, wherein the number of toroid balloons comprises in the intermediate section is greater than the number of toroid balloons of the proximal section and/or of the distal section.

Example 86. The method of any example herein, particularly any one of examples 82 to 85, wherein each of the proximal section and the distal section comprises a single one of the plurality of toroid balloons.

Example 87. The method of any example herein, particularly any one of examples 62 to 85, wherein each of the plurality of toroid balloons defines a cavity radial dimension and a cavity axial dimension at a cross-section thereof when inflated, and wherein the cavity radial dimension of at least one of the plurality of toroid balloons is different than the cavity axial dimension.

Example 88. The method of any example herein, particularly example 87, wherein the cavity axial dimension of at least one of the toroid balloons of the intermediate section is greater than the cavity radial dimension.

Example 89. The method of any example herein, particularly example 87, wherein each toroid balloon defines a cavity radial dimension which is greater than a cavity axial dimension.

Example 90. The method of any example herein, particularly example 89, wherein the cavity radial dimension is at least two times greater than the cavity axial dimension.

Example 91. The method of any example herein, particularly any one of examples 62 to 90, wherein at least one of the at least one inflation tube extends through at least one of the plurality of toroid balloons.

Example 92. The method of any example herein, particularly example 91, wherein the toroid balloons comprise axially oriented openings formed at proximal sides of balloon walls thereof.

Example 93. The method of any example herein, particularly example 92, wherein the toroid balloons further comprise axially oriented openings formed at a distal side of the balloon walls, which are aligned with the corresponding axially oriented openings formed at the proximal sides.

Example 94. The method of any example herein, particularly any one of examples 62 to 93, wherein the balloon assembly further comprises a flexible valve disposed within the perfusion lumen, the flexible valve comprising:
a valve base attached at an outer circumference thereof to at least one of the plurality of toroid balloons; and
a flexible funnel defining a channel extending proximally from a funnel inlet at the valve base to a funnel outlet and defining a funnel axis therealong;
and wherein the method further comprises:
aligning the funnel axis parallel to a longitudinal axis of the balloon assembly when blood flows in a proximally oriented direction, thereby permitting blood to pass through the channel; and
folding the flexible funnel in response to backflow in the distal direction, such that the funnel axis is angled relative to the longitudinal axis, thereby preventing blood from flowing back in the distal direction through the channel.

Example 95. The method of any example herein, particularly example 94, wherein the valve base comprises a flexible membrane.

Example 96. The method of any example herein, particularly example 94 or 95, wherein the flexible funnel is integrally formed with the valve base.

Example 97. The method of any example herein, particularly any one of examples 94 to 96, wherein the funnel outlet is narrower than the funnel inlet.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

What is claimed is:

1. A delivery apparatus comprising:
a balloon assembly comprising a plurality of toroid balloons, the plurality of toroid balloons collectively define a perfusion lumen along a longitudinal axis of the balloon assembly, wherein each toroid balloon in configured to transition between a deflated state and an inflated state, and wherein each toroid balloon comprises:
a balloon wall enclosing a cavity; and
at least one balloon opening extending through a thickness of the balloon wall; and
at least one inflation tube comprising at least one tube opening which is in fluid communication with the cavity of at least one of the plurality of toroid balloons;
wherein, when the toroid balloons are in the inflated state, the perfusion lumen is configured to allow flow therethrough.

2. The delivery apparatus of claim 1, wherein at least one of the at least one inflation tube is in fluid communication with the cavities of more than one of the plurality of toroid balloons.

3. The delivery apparatus of claim 1, wherein the at least one inflation tube comprises a plurality of inflation tubes.

4. The delivery apparatus of claim 1, wherein at least one of the at least one inflation tube extends through the perfusion lumen.

5. The delivery apparatus of claim 1, wherein the perfusion lumen defines a perfusion lumen diameter which is greater in the inflated state than in the deflated state.

6. The delivery apparatus of claim 1, wherein each toroid balloon defines a toroidal outer diameter which is greater in the inflated state than in the deflated state.

7. The delivery apparatus of claim 6, wherein at least two of the toroid balloons define different toroidal outer diameters in the inflated state.

8. The delivery apparatus of claim 1, wherein at least one of the at least one inflation tube extends through at least one of the plurality of toroid balloons.

9. The delivery apparatus of claim 1, further comprising a flexible valve disposed within the perfusion lumen and comprising:
a valve base attached at an outer circumference thereof to at least one of the plurality of toroid balloons; and
a flexible funnel defining a channel extending proximally from a funnel inlet at the valve base to a funnel outlet and defining a funnel axis therealong;
wherein the flexible valve is configured to transition between an open state in which proximally oriented flow can pass through the channel, and a closed state in response to distally oriented backflow such that the flow direction changes the state of the flexible valve.

10. A delivery apparatus comprising:
a balloon assembly comprising:
a toroid balloon that defines a perfusion lumen around a longitudinal axis of the balloon assembly, wherein the toroid balloon in configured to transition between a deflated state and an inflated state, and comprises:
a balloon wall enclosing a cavity; and
at least one balloon opening extending through a thickness of the balloon wall;
a flexible valve disposed within the perfusion lumen and comprising:
a valve base attached at an outer circumference thereof to the toroid balloon; and
a flexible funnel defining a channel extending proximally from a funnel inlet at the valve base to a funnel outlet and defining a funnel axis therealong; and
at least one inflation tube comprising at least one tube opening exposed to and in fluid communication with the cavity;
wherein the flexible valve is configured to transition between an open state in which proximally oriented flow can pass through the channel, and a closed state in response to distally oriented backflow such that the flow direction changes the state of the flexible valve.

11. The delivery apparatus of claim 10, wherein the valve base comprises a flexible membrane.

12. The delivery apparatus of claim 10, wherein the funnel outlet is narrower than the funnel inlet.

13. The delivery apparatus of claim 10, wherein the at least one inflation tube comprises a plurality of inflation tubes.

14. The delivery apparatus of claim 10, wherein the at least one inflation tube extends through the perfusion lumen.

15. The delivery apparatus of claim 10, wherein at least one of the at least one inflation tube extends through the toroid balloon.

16. A method comprising:
delivering a balloon assembly comprising a plurality of toroid balloons in a deflated state thereof, through the vasculature of a patient to a treatment site, wherein each of the toroid balloons comprises a cavity in fluid communication with at least one inflation tube; and
inflating the toroid balloons within a patient lumen and forming a perfusion lumen configured to permit blood flow therethrough,
wherein the perfusion lumen allows blood flow at least in a proximally oriented direction.

17. The method of claim 16, wherein inflating the toroid balloons to permit blood to flow through the perfusion lumen is performed without rapid pacing.

18. The method of claim 16, wherein each toroid balloon defines a toroidal outer diameter, and wherein inflating the toroid balloons comprises increasing the toroidal outer diameters such that outer sides of balloon walls of at least some of the toroid balloons press against an inner surface of the patient lumen.

19. The method of claim 16, wherein each toroid balloon defines a toroidal outer diameter, and wherein inflating the toroid balloons comprises increasing the toroidal outer diameters such that outer sides of balloon walls of at least some of the toroid balloons press against an inner surface of a prosthetic device disposed around the balloon, thereby expanding the prosthetic device against an inner wall of the patient lumen.

20. The method of claim 16, wherein the balloon assembly further comprises a flexible valve disposed within the perfusion lumen, the flexible valve comprising:
a valve base attached at an outer circumference thereof to at least one of the plurality of toroid balloons; and
a flexible funnel defining a channel extending proximally from a funnel inlet at the valve base to a funnel outlet and defining a funnel axis therealong;
and wherein the method further comprises:
aligning the funnel axis parallel to a longitudinal axis of the balloon assembly when blood flows in a proximally oriented direction, thereby permitting blood to pass through the channel; and
folding the flexible funnel in response to backflow in the distal direction, such that the funnel axis is angled relative to the longitudinal axis, thereby preventing blood from flowing back in the distal direction through the channel.
